SEQUENCE LISTING (5 sequences)

SEQ ID NO: 1 - DNA, 240 bp, Homo sapiens
cccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaactacaagcgccaggcacaggctgaccgagtgagcctgcggaacctgcgcggctactacaaccagagcgaggacggtgagtgaccccggcccggggcgcagg

SEQ ID NO: 2 - DNA, 240 bp, Homo sapiens
cccgcttcatctcagtgggctacgtggacgacacgcagttcgtgcggttcgacagcgacgccgcgagtccgagaggggagccccgggcgccgtgggtggagcaggaggggccggagtattgggaccgggagacacagaagtacaagcgccaggcacaggctgaccgagtgagcctgcggaacctgcgcggctactacaaccagagcgaggacggtgagtgaccccggcccggggcgcagg

SEQ ID NO: 3 - DNA, 240 bp, Homo sapiens
cccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacggtgagtgaccccggcccggggcgcagg

SEQ ID NO: 4 - DNA, 20 bp, Artificial sequence
  Synthetic oligonucleotide
tcatctcagtgggctacgtg

SEQ ID NO: 5 - DNA, 19 bp, Artificial sequence
  Synthetic oligonucleotide
cgtcctcgctctggttgta

(19) United States
(12) Patent Application Publication
(10) Pub. No.: US 2012/0172240 A1
(43) Pub. Date: Jul. 5, 2012

(54) GENETIC POLYMORPHISMS ASSOCIATED WITH ADVANCED LUNG DISEASES

(76) Inventors: Jianmin Xue, Pittsburgh, PA (US); Marjorie P. George, Pittsburgh, PA (US); Steven R. Duncan, Pittsburgh, PA (US)

(21) Appl. No.: 13/333,214

(22) Filed: Dec. 21, 2011

Related U.S. Application Data
(63) Continuation of application No. PCT/US10/39682, filed on Jun. 23, 2010.
(60) Provisional application No. 61/219,671, filed on Jun. 23, 2009.

(51) Int. Cl. C12Q 1/68; C40B 30/00; C40B 40/06; G01N 33/566

(52) U.S. Cl. 506/7; 436/501; 435/6.12; 506/16

(57) ABSTRACT

The present invention relates to methods and compositions that may be used to predict the risk of an individual, for example a smoker, for developing chronic obstructive pulmonary disease (“COPD”), emphysema or idiopathic pulmonary fibrosis (“IPF”).

### CROSS-REFERENCE TO RELATED APPLICATION

The present application is a continuation of International Patent Application No. PCT/US2010/039682 filed Jun. 23, 2010 and claims priority to U.S. Provisional Application No. 61/219,671 filed Jun. 23, 2009; the contents of both of these priority applications are hereby incorporated by reference in their entireties herein.

### GRANT INFORMATION

This invention was made with government support under grants 1RO1HL073241, 1RO1AR050840 and IP50 HL084948 awarded by the United States National Institutes of Health. The government has certain rights in the invention.

### 1. INTRODUCTION

The present invention relates to methods and compositions that may be used to predict the risk of an individual, for example a smoker, for developing chronic obstructive pulmonary disease (“COPD”), emphysema or idiopathic pulmonary fibrosis (“IPF”).

### 2. BACKGROUND OF THE INVENTION

Chronic obstructive pulmonary disease (COPD) is a leading cause of death in the U.S., and a major world-wide health problem. The predominant risk factor for COPD is tobacco smoking. Nonetheless, genetic factors are undoubtedly important, since only a fraction of smokers develop clinically significant COPD, and familial clustering of cases is evident. It has been shown that adaptive immune processes are active among afflicted patients, including reactivity against self-antigens (i.e., autoimmunity). Adaptive immune responses, and autoimmune processes in particular, are under the influence of genetic elements. Numerous genes of the major histocompatibility complex (MHC) on chromosome 6p21.31 are known to be immunomodulatory. The most widely recognized genes within this region are human leukocyte antigens (HLA).

Idiopathic pulmonary fibrosis (IPF) is a chronic fibroproliferative lung disease that afflicts ˜40,000 patients in the U.S. each year (31). IPF typically manifests with inexorable pulmonary restriction and hypoxemia, resulting in progressive exercise limitation and dyspnea. The disease has a grim prognosis, with a median survival of ˜3 years after diagnosis, although courses can be highly variable. No medical treatments have yet been shown to alter the natural history of this disease (31,32). No currently available tests enable accurate prediction of IPF progression in individual patients, possibly excepting extrapolations based on changes of clinical variables with replicate determinations over several months or more (33).

The etiology of IPF is enigmatic. Although not widely considered an immunologic disorder (32), several studies have shown that adaptive immune responses are often active in IPF patients, as evidenced by the frequent presence of IgG autoantibodies, increased productions of lymphocyte-derived inflammatory mediators, and abnormal extents of T-cell activation and clonal proliferations (34-44). Activated CD4 T-cells may also infiltrate into IPF lungs prior to development of symptoms (45), and pro-inflammatory dendritic cells accumulate in the pulmonary parenchyma of advanced cases (46). Moreover, a protein(s) uniquely present in IPF lungs induces proliferation of autologous CD4 T-cells from these patients indicating the presence of an intrapulmonary antigen (44). Descriptions of familial disease clusters also implicate a role of genetic mechanisms in IPF (45,47).

Specific HLA alleles are often associated with various immunologic diseases. Two distinct mechanisms may account for these associations. HLA molecules are requisite effectors for presentation of peptide antigens to the T-cells that initiate adaptive immune responses, but each distinct HLA allele has a unique, restricted, peptide binding motif. Hence, the genetically-determined presence of specific (“permissive”) HLA alleles may result in presentations of disease-causing antigens to T-cells. Although important for host defense, in some cases these immune responses may be deleterious if, as an example, the antigen is a self-protein (autoantigen), or one that evokes a cross-response to a self-protein (epitope mimicry). In contrast, humans lacking these specific, permissive HLA alleles do not present those particular antigen epitopes, and do not initiate the deleterious response(s). Alternatively, relative overexpressions of particular HLA alleles in a disease cohort may be unrelated to antigen presentations per se, but are instead genetic “markers” indicating the presence of other important immunomodulatory genes that flank, and are in linkage disequilibrium (LD), with these HLA. Thus, identification of a distinct disease-susceptibility region within the MHC complex that includes multiple linked genes (e.g., a haplotype) may be necessary to unravel pathogenic processes.

### 3. SUMMARY OF THE INVENTION

The present invention relates to methods and compositions for evaluating the risk of an individual for developing COPD or emphysema. It is based, at least in part, on a comparison of HLA alleles of end-stage COPD patients to the alleles of a control cohort of subjects with extensive smoking histories but normal spirometry which found that HLA-Cw*0702 is strongly associated with this disease. Further association has been found between COPD and the alleles HLA-B*0702 and/or DRB1*1501. These same alleles have also been associated with IPF. In contrast, expressions of certain other alleles, for example, Cw*05, Cw*08 and Cw*12, appear to be “protective” for COPD. These data show that one or more genes within or in proximity to this distinct HLA region(s) are important determinants of COPD susceptibility.

For example, certain non-limiting embodiments of the invention are based on the discovery that Cw*07 is the single most prevalent HLA allele among COPD patients. Accordingly, the present invention provides for a method of determining that a subject is at higher risk than the general population for developing COPD or emphysema, comprising testing the individual for the presence of the Cw*07 HLA allele, wherein, if the allele is present, the subject is at higher risk for developing COPD or emphysema. In such cases, it may be desirable to recommend a further procedure, such as a chest X-ray and/or a pulmonary function test such as spirometry, and/or to counsel the subject that smoking or other exposures to lung damaging agents could be dangerous. Further, it may be desirable to recommend that a subject identified as being at high risk receive at least annual vaccination against influenza or pneumonia. The further presence of HLA-B*0702 and/or DRB1*1501 alleles would further corroborate the higher risk.

Further non-limiting embodiments of the invention are based on the discovery that Cw*0702 is the more prevalent Cw*07 HLA allele among COPD patients. Accordingly, the present invention provides for a method of determining that a subject is at higher risk than the general population for developing COPD or emphysema, comprising testing the individual for the presence of the Cw*0702 HLA allele, wherein, if the allele is present, the subject is at higher risk for developing COPD or emphysema. In such cases, it may be desirable to recommend a further procedure, such as a chest X-ray and/or a pulmonary function test such as spirometry, and/or to counsel the subject that smoking or other exposures to lung damaging agents could be dangerous. Further, it may be desirable to recommend that a subject identified as being at high risk receive at least annual vaccination against influenza or pneumonia. The further presence of HLA-B*0702 and/or DRB1*1501 alleles would further corroborate the higher risk.

In other non-limiting embodiments, the present invention provides for a method of determining that a subject is at higher risk than the general population for developing COPD or emphysema, comprising testing the individual for the presence of the HLA-B*07, HLA-B*0702, HLA-A*03 and/or DRB1*1501 HLA allele, wherein, if the allele is present, the subject is at higher risk for developing COPD or emphysema. In such cases, it may be desirable to recommend a further procedure, such as a chest X-ray and/or a pulmonary function test such as spirometry, and/or to counsel the subject that smoking or other exposures to lung damaging agents could be dangerous. Further, it may be desirable to recommend that a subject identified as being at high risk receive at least annual vaccination against influenza or pneumonia.

In certain non-limiting embodiments, the present invention provides for a kit for determining the risk of a subject for developing COPD or emphysema, comprising a means for detecting the presence of one or more allele selected from the group consisting of Cw*07, Cw*0702, HLA-B*07, HLA-B*0702, HLA-A*03 and DRB1*1501, Said kit may include, for example, primers for detecting said allele(s) using polymerase chain reaction, or antibodies specific for said alleles.

The present invention also relates to methods and compositions for evaluating the risk of an individual for developing idiopathic pulmonary fibrosis (IPF). It is based, at least in part, on a comparison of HLA alleles of end-stage IPF patients to the alleles of a control cohort of normal healthy subjects which found that DRB1*1501 is strongly associated with this disease, although there are also associations between IPF and Cw*07 and B*07. Without being bound by any particular theory, the associations between IPF and Cw*07 and B*07 may be explained by strong linkage disequilibrium with DRB1*1501.

The present invention provides for a method of determining that a subject is at higher risk than the general population for developing IPF, comprising testing the individual for the presence of a DRB1*15, Cw*07, B*07, or DQB1*0602, and most preferably a DRB1*1501, HLA allele, wherein, if said one or more allele is present, the subject is at higher risk for developing IPF. In such cases, it may be desirable to recommend a further procedure, such as a chest X-ray and/or a pulmonary function test such as spirometry, and/or to counsel the subject that smoking or other exposures to lung damaging agents could be dangerous. Further, it may be desirable to recommend that a subject identified as being at high risk receive at least annual vaccination against influenza or pneumonia. The presence of more than one of DRB1*15, DRB1*1501, Cw*07, B*07, or DQB1*0602 would further corroborate the higher risk.

In other non-limiting embodiments, detecting the presence of the DRB1*15 HLA allele indicates that the subject is at higher risk for developing IPF than a person without the allele.

Further non-limiting embodiments of the invention are based on the discovery that DRB1*1501 is the more prevalent DRB1*15 HLA allele among IPF patients. Accordingly, the present invention provides for a method of determining that a subject is at higher risk than the general population for developing IPF, comprising testing the individual for the presence of the DRB1*1501 HLA allele, wherein, if the allele is present, the subject is at higher risk for developing IPF. In such cases, it may be desirable to recommend a further procedure, such as a chest X-ray and/or a pulmonary function test such as spirometry, and/or to counsel the subject that smoking or other exposures to lung damaging agents could be dangerous. Further, it may be desirable to recommend that a subject identified as being at high risk receive at least annual vaccination against influenza or pneumonia. The further presence of DQB1*0602 allele would further corroborate the higher risk.

In certain non-limiting embodiments, the present invention provides for a kit for determining the risk of a subject for developing IPF, comprising a means for detecting the presence of one or more allele selected from the group consisting of DRB1*15, DRB1*1501, Cw*07, Cw*0702, B*07, B*0702, and DQB1*0602, preferably including at least DRB1*1501. Said kit may include, for example, primers for detecting said allele(s) using polymerase chain reaction, or antibodies specific for said alleles.

In other non-limiting embodiments, the present invention provides for methods of treating a subject or individual, for example, a human or other mammal, that has been diagnosed with COPD, emphysema or IPF, or that expresses a COPD HLA allele or IPF HLA allele, by administering an agent to the individual or subject in an amount effective to inhibit, reduce or block the expression of a COPD HLA allele or an IPF HLA allele.

### 4. BRIEF DESCRIPTION OF THE DRAWINGS

FIG. 1 Shows A) Smoke Controls positive for Cw*0702 (closed columns, n=13) have greater prevalence of radiographic emphysema (OR: 3.5; 95% CI: 1.0-12.5), and greater aggregate emphysema scores than Smoke Controls (open columns, n=69) who are negative for this allele. P values are denoted above or proximate to brackets. B) Demographic characteristics of the Cw*0702⁺ Smoke Controls are similar to those of the Cw*0702⁻ subjects, except for greater ages among the latter. PY denotes cumulative pack-years of smoking, CS denotes subjects currently smoking.

FIG. 2 Shows gene alignments of the HLA region on chromosome 6p21.3. The major HLA loci are denoted in red. This is a very schematic representation, whereas a complete description of genes within this region is available on GenBank and other sources (27). A small number of the many other genes with immunomodulatory function within this region are denoted in blue. “C”; complement proteins, TNF; tumor necrosis factor, MICB and MICA; MHC-class I-chain repeated genes B and A, respectively. No functioning genes are known to exist between HLA-B and -Cw.

FIG. 3 Prevalence of risk (Cw*0702), protective (Cw*05, Cw*08, Cw*12), and other Cw alleles in COPD and Smoke Controls (SC), open and closed columns, respectively. Odds ratio (OR) and 95% confidence intervals denote the association between the presence of a Cw*0702 allele (irrespective of heterozygosity for a protective allele) and COPD among these stratified subjects.

FIG. 4 Shows the demographics of study subjects. FEV₁: forced expiratory volume in one second; % predicted: percentages of normal, predicted values (34); DL_{co}: single breath diffusing capacity for carbon monoxide (35). Emphysema was defined as Likert scores >0. * p<0.0001.

FIG. 5a-5c Shows the HLA-A-HLA-DRB*15 Allele prevalence among study subjects. Values listed are allele prevalences among end-stage (COPD) patients and SC (Smoke Controls). Alleles denoted by bold font are in LD with Cw*0702. P values are for intergroup comparisons between SC and COPD. Pc denotes alpha values corrected for multiple comparisons (Bonferroni). NS; not significant, NA; not applicable.

FIG. 6. Shows the polymerase chain reaction specific sequence primer method for HLA typing. Lanes 1 to 23 contain PCR products of 23 different primer mixes corresponding to specific HLA-Cw sequences. Lane 24 is a negative control. Internal positive control primer pairs amplify segments of the human growth hormone gene. The two different control primer pairs give rise to either positive control bands of 1070 bp, or 800 bp. HLA alleles are assigned by the presence of specific PCR product(s), discriminated here by electrophoresis in 2% agarose gels with ethidium bromide staining. Different amplification patterns and product lengths denote the various alleles. As an example here, lanes 6 (125 bp), 11 (340 bp), 13 (130 bp), 18 (190 bp) and 21 (325 bp) denote the Cw*04 allele. Lanes 9 (245 bp) and 23 (225 bp) here denote the Cw*07 allele. Thus, this specimen is heterozygous for Cw*04 and Cw*07 alleles.

FIG. 7 Shows a comparison of the HLA alleles Cw*0701, Cw*0704 and Cw*0702 (SEQ ID NOS.; 1-3). Segments with turquoise highlighting correspond to primer sequences. Yellow highlighting denotes the by substitution that distinguishes Cw*0701 (C) from Cw*0702 (G). Among Caucasians, these two alleles comprise ˜97% of the Cw*07 among Caucasians (ref. 17). In turn, these alleles can be distinguished from the relatively rare Cw*0704 (˜3% of Caucasians with Cw*07) by the G-C substitution denoted by red highlighting.

FIG. 8 Shows the polymerase chain reaction restriction fragment length polymorphism (PCRRFLP) method to detect HLA-DRB1*15 (see also reference 18). Genomic DNA was amplified using DR2-specific primers. The PCR product was digested with Fok I and Sau96I to differentiate DR15 from DR16. Products were separated by electrophoresis on 12% acrylamide gels and visualized with ethidium bromide. Complete digestions of the amplified DNA with both enzymes identifies DR15 (samples 1, 2, 4); no digestion identifies the presence of DR16 (sample 3). Numbers below the figure indicate digestion pattern; 0=no digestion; 1=complete digestion.

FIG. 9 Shows pulmonary function of smoke controls stratified by the presence of Cw*0702. There were no significant differences of pulmonary function among the Smoke Controls with and without Cw*0702.

FIG. 10 Shows the prevalence of risk or protective HLA-Cw alleles (or none of these) among COPD and control subjects. Those few subjects with both risk and protective alleles were stratified in the risk category, based on assumptions that the former is dominant. OR and CI here denotes association of a protective Cw allele (vs. CW*0702 or neither) with the presence of disease. Conversely expressed, the presence of Cw*0702 vs. either a protective allele or an irrelevant allele, yields an OR 9.0; 95% CI: 3.0-25.0 (p<0.0001) for an association with end-stage COPD.

### 5. DETAILED DESCRIPTION OF THE INVENTION

The present invention is based on the discovery that the presence of certain HLA alleles in a subject indicate that the subject is at higher risk for developing COPD, emphysema or IPF. In cases where such biomarkers are detected, it may be desirable to recommend a further procedure, such as a chest X-ray and/or a pulmonary function test such as spirometry, and/or to counsel the subject that smoking or other exposures to lung damaging agents could be dangerous. Further, it may be desirable to recommend that a subject identified as being at high risk receive at least annual vaccination against influenza or pneumonia.

For clarity and not by way of limitation, this detailed description is divided into the following sub-portions:

### (i) Definitions;

(ii) HLA alleles;
(iii) Assays for the detection of HLA alleles;
(iv) Methods of use and treatment; and

### (v) Kits.

### 5.1. DEFINITIONS

The terms “COPD HLA allele” or “COPD biomarker” refers to one or more HLA alleles that indicate a subject has COPD or emphysema, or has a higher risk for developing COPD or emphysema, when the HLA allele is present in the individual. In one non-limiting embodiment, the COPD HLA alleles are the Cw*07, Cw*0702, HLA-B*07, HLA-B*0702, HLA-A*03 and DRB1*1501 HLA alleles.

The terms “protective COPD HLA allele” or “protective COPD biomarker” refers to one or more HLA alleles that indicate a subject has a reduced risk for developing COPD or emphysema when the HLA allele is present in the individual. In one non-limiting embodiment, the protective COPD HLA alleles are the Cw*05, Cw*08 and Cw*12 HLA alleles.

The terms “IPF HLA allele” or “IPF biomarker” refers to one or more HLA alleles that indicate a subject has IPF, or has a higher risk for developing IPF, when the HLA allele is present in the individual. In one non-limiting embodiment, the IPF HLA alleles are the DRB1*15, DRB1*1501, Cw*07, Cw*0702, B*07, B*0702 and DQB1*0602 HLA alleles.

The present invention also encompasses DNA segments that are complementary, or essentially complementary, to the HLA alleles described herein. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a specified nucleic acid segment, under relatively stringent conditions as understood in the art.

Hybridizing segments may be relatively short nucleic acids, often termed oligonucleotides. Sequences of at least 10 bases long, for example, sequences of at least 17 or at least 22 bases long, should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of any number from 8 to 100 or more base pairs will be used, although others are contemplated. Longer polynucleotides are contemplated as well. Such oligonucleotides will find use, for example, as probes in Southern and Northern blots and as primers in amplification reactions.

### 5.2. HLA ALLELES

The present invention is based at least in part on the identification of HLA alleles that indicate a higher risk of developing COPD, emphysema or IPF when the HLA alleles are present in a subject, such as, for example, a human subject. The present invention is also based in part on the identification of HLA alleles that indicate a reduced risk for developing COPD or emphysema when the HLA alleles are present in a subject.

In one non-limiting embodiment, the COPD HLA alleles include, but are not limited to, the Cw*07, Cw*0702, HLA-B*07, HLA-B*0702, HLA-A*03 and DRB1*1501 HLA alleles.

In one non-limiting embodiment, the COPD HLA allele is the Cw*0702 HLA allele.

In another non-limiting embodiment, the IPF HLA alleles include, but are not limited to, the Cw*07, Cw*0702, B*07, B*0702, DRB1*15, DRB*1501 and DQB1*0602 HLA alleles, and most preferably include the DRB1*1501 HLA allele.

In other non-limiting embodiments, the protective COPD HLA alleles include, but are not limited to, the Cw*05, Cw*08 and Cw*12.

### 5.3. ASSAYS FOR THE DETECTION OF HLA ALLELES

According to the present invention, certain HLA alleles are present in individuals with COPD, emphysema or IPF. Detection of these HLA alleles can be used to detect or diagnose COPD, emphysema or IPF in a subject, such as, for example, a human subject. Detection of HLA alleles associated with COPD, emphysema or IPF is also helpful to identify subjects at risk for developing COPD, emphysema or IPF, and initiating a preventative or treatment regimen. Assays such as RT-PCR, PCR, qPCR, DNA and RNA sequencing, microarray analysis and any other genome-based analyses known in the art, along with any immunoassays known in the art, may be used to detect a COPD HLA allele, protective COPD allele or an IPF HLA allele in a sample from an individual, for example, plasma, serum, cerebrospinal fluid, sputum, saliva, breast milk, tears, bile, semen, vaginal secretion, amniotic fluid, urine, stool, leukocytes, bone marrow cells, buccal cells, fibroblasts and lung or other tissue biopsies. In addition, such analyses may be qualitative or quantitative.

In humans, HLA alleles may be detected individually or in combination to provide a diagnostic evaluation of COPD, emphysema or IPF.

### PCR Detection of HLA Alleles

In certain embodiments of the present invention, HLA alleles are detected by polymerase chain reaction (PCR) or reverse transcriptase-polymerase chain reaction (RT-PCR).

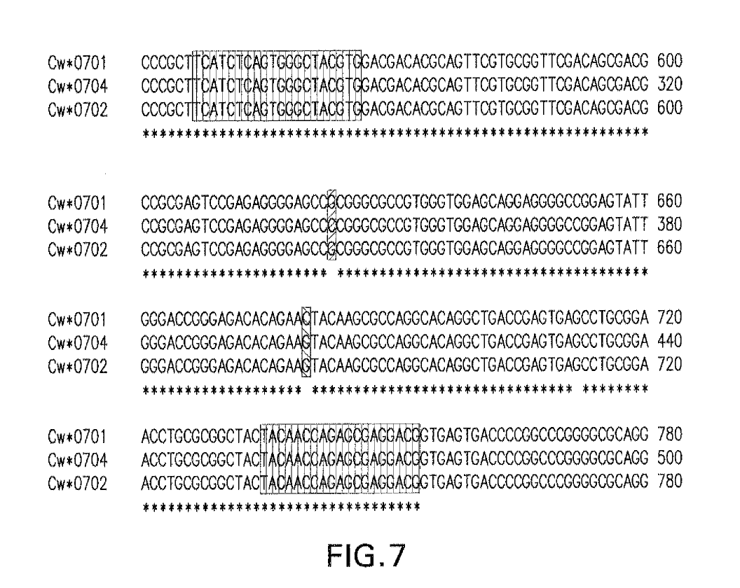

In one non-limiting embodiment, the HLA allele is amplified from genomic DNA of a patient. HLA alleles may be amplified through PCR by using at least one set of primers for each HLA allele. Sets of primers flanking an HLA region to be sequenced may be designed based on HLA sequences available and known in the art (see, Genbank Accession numbers DQ359691, D49819, D38526, AJ293016, FJ515904, Z49112, EF694833, AM182459, FJ821318, AM904554, AM746337, AF436098, AJ292075, AJ309047, U49905, U49904, EU305401, FN806804, FN806803, AM849481, FM955270, M16957, M17378, M20430, L78169, AY375871, AY375870, L34105, FM865852, AJ420244, FJ750479, AF016304, AF016303, FJ825144, FJ868794, FN568089, AM180647, AJ579649, AJ579648, AJ579647, FJ811899, and the allele reports for C*07:02:01:01, B*07:02:01, DRB1*15:01:01:01, and DQB1*06:02:01 in the EMBL-EBI IMGT/JLA database (72-74) and see FIG. 7). In non-limiting embodiments of the invention, each primer may be at least about 10, or at least about 12, or at least about 15, or at least about 20 bases long, and may have a total length of between 10 and 100, or between 10 and 50, or between 10 and 30, or between 10 and 25, or between 12 and 100, or between 12 and 50, or between 12 and 30, or between 12 and 25, bases long, and be complementary to at least about 10, or at least about 12, or at least about 15, or at least about 20, or at least about 25 bases of the HLA allele it is intended to amplify. Following PCR amplification, the PCR amplification products can be sequenced using standard techniques known in the art. In accordance with the present invention, production of an HLA allele PCR amplification product indicates that an individual is at risk for developing COPD, emphysema or IPF.

In other non-limiting embodiments, an HLA allele transcription product, or mRNA, may be amplified using RT-PCR.

In one non-limiting embodiment, the oligonucleotide primers used for amplifying an HLA allele (e.g., PCR and RT-PCR) are complementary to a wild type HLA allele sequence. In other embodiments, the oligonucleotide primers are complementary to a mutant HLA allele sequence.

In a further non-limiting embodiment, the HLA allele nucleic acid (e.g. genomic DNA or RNA) can be derived from tissues, cells and/or cells in biological fluids from a mammal or human to be tested.

Standard cloning and molecular biology techniques are well known in the art and unless otherwise noted, they can be carried out according to various techniques described by Sambrook et al. (2001) Molecular Cloning: A Laboratory Manual. 3rd Ed. Cold Spring Harbor Laboratory Press: Cold Spring Harbor, N.Y., or according to various techniques described in Ausubel et al. eds. (2005) Current Protocols in Molecular Biology. John Wiley and Sons, Inc.: Hoboken, N.J. Each of these references is hereby incorporated by reference in their entireties.

### Quantitative PCR

According to the present invention, individuals may be screened for the presence of an HLA allele through the use of quantitative polymerase chain reaction (qPCR), or quantitative reverse transcriptase-polymerase chain reaction (qRT-PCR), which utilizes competitive techniques employing an internal homologous control that differs in size from the target, for example, by a small insertion or deletion. Non-competitive and kinetic quantitative PCR or RT-PCR may also be used. Experiments may combine real-time, kinetic PCR or RT-PCR detection together with an internal homologous control that can be simultaneously detected alongside the target sequences. In one non-limiting embodiment, real time quantitative PCR may provide the capability of measuring the level of an HLA allele gene product amplified through PCR. In another non-limiting embodiment, quantitative PCR may require only a nominal amount of a sample to perform such experiments.

Quantitative amplification is based on the monitoring of a signal (e.g., fluorescence of a probe) representing copies of a template in cycles of an amplification (e.g., PCR) reaction. In the initial cycles of the PCR, a very low signal is observed because the quantity of the amplification product formed does not support a measurable signal output from the assay. After the initial cycles, as the amount of formed amplification product increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., U.S. Pat. Nos. 6,180,349; 6,033,854; and 5,972,602.

### Detection of HLA Alleles Using Nucleic Acid Microarrays

In one non-limiting embodiment of the invention, nucleic acid microarrays, or gene chip technology, may be used to screen and identify patients who carry a COPD HLA allele, protective COPD HLA allele or an IPF HLA allele (see, e.g., U.S. Pat. No. 7,455,975). As used herein, a “microarray” is an array of distinct polynucleotides, oligonucleotides, polypeptides, peptides, or antibodies affixed to a substrate, such as paper, nylon, or other type of membrane; filter; chip; glass slide; or any other type of suitable support.

In one non-limiting embodiment, the microarray technology involves the positioning of highly condensed and ordered arrays of nucleic acid probes, for example, DNA oligonucleotides, on a substrate, for example, a glass slide or nylon membrane. Each oligonucleotide may comprise a nucleotide sequence that is complementary to a portion of an HLA allele, wherein the oligonucleotide can be placed on a single glass slide or nylon membrane. For example, and not by way of limitation, up to 50,000 DNA fragments, may be placed on a single glass slide and up to 5,000 placed on a nylon membrane. The resulting microarrays can then be used to screen for the presence an HLA allele transcription product expressed in a sample to be screened.

In another non-limiting embodiment, a nucleic acid microarray may be utilized by preparing labeled nucleic acid from a sample to be screened, and hybridizing such labeled nucleic acid with the array. In addition, labeled nucleic acid of a designated control sequences may be prepared (or in the event that the array is sold as part of a kit, could be supplied to the user). Radioactive, colorimetric, chemiluminescent or fluorescent tags may be used for labeling of nucleic acid sequences from the sample and for the control. Numerous techniques for scanning arrays, detecting fluorescent, chemiluminescent, or colorimetric output, are known in the art and may be used for detecting hybridization of a nucleic acid from a test sample to the microarray. For example, a low-cost, high-throughput fluorescent microarray scanning system (ScanArray®, PerkinElmer Life And Analytical Sciences, Inc., Waltham, Mass., USA), or a colorimetric microarray scanner (ArrayIt® SpotWare™, TeleChem International, Inc., Sunnyvale, Calif., USA) may be used. Numerous protocols for the preparation of labeled nucleic acid sequences are publicly available and may be used.

The present invention contemplates the preparation of one or more specialized microarrays (e.g., oligonucleotide microarrays or cDNA microarrays) comprising one or more polynucleotides encoding one or more HLA allele or complementary sequences, or fragments thereof. In accordance with this aspect of the invention, the oligonucleotide sequences or cDNA sequences include any of the disclosed HLA alleles or fragments or combinations thereof, which are expressed in cells of an individual who is at risk for developing COPD, emphysema or IPF, and are contained on a microarray, e.g., a oligonucleotide microarray or cDNA microarray in association with, or introduced onto, any supporting materials, such as glass slides, nylon membrane filters, glass or polymer beads, or other types of suitable substrate material.

Methods for producing and using DNA microarrays are well known in the art. For example, to determine gene expression using microarray technology, polynucleotides, e.g., RNA, DNA, or cDNA, are isolated from a biological sample, e.g., cells expressing an HLA allele. The isolated nucleic acid is detectably labeled, e.g., by fluorescent, enzyme, or chemiluminescent label, and applied to a microarray, e.g., one or more nucleic acid microarrays provided by this invention which comprises, for example, oligonucleotides complimentary to the labeled cellular derived nucleic acid applied to the microarray. The array is then washed to remove unbound material and visualized by staining or fluorescence, or other means known in the art depending on the type of label utilized.

### Detection of HLA Alleles Using Nucleic Acid Sequencing

In another non-limiting embodiment, an individual may be screened for an HLA allele through sequencing (i.e. determining the nucleotide sequence of a given DNA or RNA fragment) of a genomic DNA or HLA allele expression product present in a sample taken from the individual. Any sequencing methods known in the art may be used to determine the nucleotide order of the HLA allele DNA or RNA. For example, but not by way of limitation, an HLA allele may be identified by first performing PCR and then sequencing the product of PCR to determine the specific allele (see Listgarten et al., 2008, PloS omput. Biol. 4(2):e1000016).

For example, and not by way of limitation, chain terminator sequencing (i.e. Sanger sequencing) may be used to sequence the HLA allele expression product, wherein extension of a polynucleotide is initiated at a specific site on the template HLA allele nucleic acid (e.g., DNA) by using a short oligonucleotide primer complementary to the template at that region. The classical chain-termination method requires a single-stranded DNA template, a DNA primer, a DNA polymerase, radioactively or fluorescently labeled nucleotides, and modified nucleotides that terminate DNA strand elongation (e.g., di-deoxynucleotides). The DNA sample may be divided into four separate sequencing reactions, containing all four of the standard deoxynucleotides (dATP, dGTP, dCTP and dTTP) and the DNA polymerase. One of the four dideoxynucleotides (ddATP, ddGTP, ddCTP, or ddTTP) are added to each of the four reactions, which are the chain-terminating nucleotides, lacking a 3′-OH group required for the formation of a phosphodiester bond between two nucleotides, thus terminating DNA strand extension and resulting in various DNA fragments of varying length.

Newly synthesized and labeled DNA fragments are heat denatured, and separated by size by, for example, gel electrophoresis, with each of the four reactions run in one of four individual lanes of the gel (lanes A, T, G, C). The DNA bands may be visualized by autoradiography or UV light, and the DNA sequence can be directly read off the X-ray film or gel image.

In one embodiment, the primer is labeled (e.g., a fluorescent or radioactive label). In other embodiments, the chain-terminator nucleotides are labeled, for example, in ‘dye terminator sequencing’. In dye terminator sequencing, complete sequencing may be performed in a single reaction, wherein each of the di-deoxynucleotide chain-terminators (e.g., ddATP, ddGTP, ddCTP, and ddTTP) are labeled with a separate fluorescent dye which fluoresces at a different wavelength. The sequence of the template may be determined by separating the synthesized polynucleotide by size and determining the order of the dye signals exhibited by the reaction products.

The sequencing of a nucleic acid sample (i.e. determining the nucleotide order of a given DNA or RNA fragment) is not limited to any one technique. The present invention contemplates the use of any sequencing technique known in the art.

### Immunologic Detection of HLA Alleles

In certain embodiments, the present invention entails the use of antibodies in the immunologic detection of protein isoforms resulting from expression of a COPD HLA allele, protective COPD HLA allele or IPF HLA allele. Immunoassays, in their most simple and direct sense, are binding assays. Certain preferred immunoassays include, but are not limited to, enzyme linked immunosorbent assays (ELISAs), Western blots and radioimmunoassays (RIA). Immunohistochemical detection using tissue sections also is particularly useful. However, it will be readily appreciated that detection is not limited to such techniques. For example, Western blotting, dot blotting, FACS analyses, and the like also may be used in connection with the present invention.

In one non-limiting embodiment, the immunological detection methods of the invention can discriminate between protein isoforms resulting from expression of a COPD HLA allele, protective COPD HLA allele or IPF HLA allele and protein isoforms resulting from expression of a non-COPD HLA allele, non-protective COPD HLA allele or non-IPF HLA allele. For example, when a sample comprises both a protein resulting from a COPD HLA allele and a protein resulting from a non-COPD HLA allele, the immunological methods of the present invention may only detect the protein resulting from the COPD HLA

### 5.4. TREATMENTS AND METHODS OF USE

In considering the potential impact of the present invention, it should be noted that COPD and IPF are global scourges, with woefully inadequate current treatments, and a complex and poorly understood pathogenesis. Although the potential importance of genetic factors in COPD is well appreciated (1,2), and it is currently a very active area of research interest, findings to date can at best only partly account for disease progression (3,4). The present application indicates that a specific region of the HLA complex, and particular HLA alleles within this region, are highly associated with the presence (or absence) of disease in many COPD or IPF patients. The impact of the invention has at least six aspects, as follows.

In a first, non-limiting embodiment, determinations of these HLA biomarkers can be useful in the care of individual smokers. Among other aspects, individualized odds ratios for eventual development of IPF, COPD and/or emphysema could be productively used by health care personnel in intense, personalized smoking cessation efforts. In addition, smokers or former smokers at high-risk for IPF or COPD (because of their HLA haplotype) could benefit from more intense or detailed routine medical surveillance (e.g., pulmonary function tests) to detect early disease.

In a second, non-limiting embodiment, individuals at high-risk, based on their HLA haplotypes, could also be specifically targeted for recruitments into long-term observational or interventional clinical trials. The latter could include studies of early interventions that might have the potential to modify intrapulmonary inflammation (e.g., inhaled corticosteroids, macrolides, statins, etc.) and perhaps favorably alter the natural history of COPD or IPF.

In a third, non-limiting embodiment, the results of these studies will build a foundation for subsequent incremental investigations to further unravel fine details of disease mechanisms. Identification of T-cell and B-cell antigens that drive the inflammation of COPD or IPF is an active topic of research.

In a fourth, non-limiting embodiment, the present findings also show certain HLA-Cw alleles are associated with lessened predilections for the development of COPD. These completely novel and important data will provide yet another avenue for subsequent incremental dissection of molecular mechanisms, by allowing focused comparisons of potentially important immunomodulatory genes in proximity to the protective alleles (e.g., Cw*05, Cw*08, Cw*12) vs. their polymorphic variants in LD with the risk-conferring HLA alleles (e.g., Cw*0702).

In a fifth, non-limiting embodiment, the present invention may also be useful to devise combinations of gene expressions, including non-HLA genes outside the HLA complex that also associate with COPD or IPF (reviewed in 3,4), for higher power prognostications or identifications of highest-risk cohorts. Thus, permutations of specific HLA allele expressions, in combinations with expressions at the other (non-HLA) genetic loci that confer risk (3,4), could prove to have even greater associations with COPD (or IPF) and/or clinical manifestations of the disease.

In a sixth, non-limiting embodiment, by providing insight into the molecular mechanisms of COPD and IPF, the present invention may be used to develop novel and more efficacious therapeutics that are specifically targeted at these disease processes.

In other non-limiting embodiments, the present invention provides for methods of treating a subject or individual, for example, a human or other mammal, that has been diagnosed with COPD, emphysema or IPF, or that expresses a COPD HLA allele or IPF HLA allele, by administering an agent to the individual or subject in an amount effective to inhibit, reduce or block the expression of a COPD HLA allele or an IPF HLA allele.

For example, the expression of a target HLA allele can be inhibited, reduced or blocked by administering an siRNA molecule, RNA interference molecule or antibody to the subject or individual in an amount effective to inhibit, reduce or block the expression of the target HLA allele. In other embodiments, the siRNA molecule, RNA interference molecule or antibody is administered to the subject or individual in an amount effective to reduce, lower or lessen one or more symptom associated with COPD, emphysema and/or IPF.

In addition to siRNA molecules, RNA interference molecules and antibodies, the agents of the present application include any compound that can reduce the expression of a COPD HLA allele or IPF HLA allele, or reduce one or more symptom of COPD, emphysema or IPF, for example, small molecules that block HLA allele expression.

### 5.5. KITS

In further embodiments, the present invention provides kits for use in detecting an HLA allele nucleic acid or protein in a biological sample. Such kits will generally comprise one or more oligonucleotides and/or antibodies that have specificity for various HLA allele nucleic acids or proteins.

In one non-limiting embodiment, a kit for detection of an HLA allele nucleic acid will comprise, in suitable container means, one or more control HLA allele nucleic acid and one or more oligonucleotide that specifically hybridizes to the HLA allele or control HLA allele nucleic acid, or region thereof, for use in PCR, RT-PCR, qPCR, qRT-PCR, microarray analysis, Southern blot analysis or its equivalent, or nucleic acid sequencing. The kit may also comprise one or more polymerase, reverse transcriptase, and nucleotide bases, wherein the nucleotide bases may be further detectably labeled.

In other non-limiting embodiments, the immunodetection kits will comprise, in suitable container means, one or more control HLA allele protein, and one or more antibodies that bind to the control HLA allele protein and proteins resulting from COPD HLA allele, protective COPD HLA allele or IPF HLA allele, and antibodies that bind to other antibodies via Fc portions.

The immunodetection reagents of the kit may include detectable labels that are associated with, or linked to, the given antibody or antigen itself. Detectable labels that are associated with or attached to a secondary binding ligand are also contemplated. Such detectable labels include, for example, chemiluminescent or fluorescent molecules (e.g., rhodamine, fluorescein, green fluorescent protein, luciferase, Cy3, Cy5, or ROX), radiolabels (e.g., ³H, ³⁵S, ³²P, ¹³¹I) or enzymes (e.g., alkaline phosphatase, horseradish peroxidase).

In one non-limiting embodiment, the kit comprises at least one pair of oligonucleotide primers that is capable of hybridizing to one or more of the following HLA alleles: Cw*07, Cw*0702, B*07, HLA-B*0702, DRB1*1501, DQB1*0602, Cw*05, Cw*08 and Cw*12.

### 6. EXAMPLES

### Example 1

### Haplotypes Associated with COPD

### Introduction

Chronic obstructive pulmonary disease (COPD) is a global scourge, with inadequate medical treatments, and a poorly understood pathogenesis (58). Although tobacco smoking is the single greatest risk factor for COPD, other disease mechanisms are also important, since only a fraction of smokers develop severe manifestations, and familial clustering of cases is evident (1,2). Active adaptive immune responses are present among those afflicted, and the magnitude of these processes often correlate with disease severity (59, 60, 11, 61, 12 and 9).

HLA allele frequencies are often abnormally distributed among patients with immunologic disorders (24, 25 and 14), but HLA characterizations of COPD populations have not been extensively pursued (28,29). It was hypothesized that relative over- or under-representations of specific HLA frequencies may also occur in COPD, and these would likely be especially evident among the most severely afflicted. Comparisons to controls with extensive smoking histories but normal spirometry (Smoke Controls) could have greater power than use of random (and mostly nonsmoking) healthy volunteers, with the assumption Smoke Controls were largely bereft of COPD susceptibility alleles (or they would have lung disease), and/or they may be “enriched” for “disease-protective” alleles.

Given considerations that CD8 T-cell infiltrations and activation are especially prominent in COPD (59, 60, 11, 61, 12 and 9), and these lymphocytes are largely HLA Class I-dependent, the HLA Class I allele frequencies of these patients were evaluated.

Alleles of the HLA-Cw locus were found to be highly associated with susceptibility for expiratory airflow obstruction (COPD) and radiographic emphysema.

### SUMMARY

### Background

Adaptive immune responses appear to be important in the pathogenesis of chronic obstructive pulmonary disease (COPD). It was hypothesized that HLA allele frequency perturbations may be present in COPD patients, as they often are among those afflicted with other immunologic diseases. Methods: HLA Class I allele frequencies of 82 patients with end-stage COPD were compared to those of 82 subjects with extensive smoking histories, but normal spirometry (Smoke Controls).

### Results

HLA-Cw*0702 was over-represented among COPD (46%) compared to Smoke Controls (16%) (OR 4.6, 95% CI: 2.2-9.5, P<0.0001). Moreover, this disease association was greater than that for HLA-A and HLA-B alleles, including those in linkage disequilibrium (LD) with Cw*0702. Cw*0702 was also associated with the radiographic presence of emphysema, independently of expiratory airflow, among the Smoke Controls (OR 3.7, 95% CI: 1.0-13.2, P<0.05). Conversely, Cw*05, Cw*08, and Cw*12 were each ˜2-fold more prevalent in Smoke Controls than among COPD: the presence of any of these alleles were associated with OR: 0.33, 95% CI: 0.17-0.64 (P=0.001) for disease. The presence of a dominant Cw*0702 vs. protective or irrelevant Cw alleles yielded OR: 11.6, 95% CI: 4.5-30.3 (P<0.0001) for COPD.

### Conclusion

HLA-Cw*0702 is associated with susceptibility for COPD among heavy smokers, whereas Cw alleles 05, 08, and 12 appear protective. These data show genetic variants conferring susceptibility and/or protection from COPD are among, or proximate to (and in LO with), the HLA-Cw locus. These findings may have importance for risk assignments of individual patients, as well as providing a focus for subsequent targeted genomic and mechanistic investigations.

### Methods

Subjects: COPD subjects consisted of patients with molecular HLA allele determinations who had lung transplantations for this disease at the University of Pittsburgh Medical Center. All fulfilled European Respiratory Society/American Thoracic Society criteria, having severe, fixed expiratory airflow obstruction attributable to smoking (≧10 pack-years), and the absence of other known causes of their lung disease (20). Characteristic airway and emphysematous abnormalities were documented in all by pre-transplant chest computerized tomography (CT) scans and lung explant histology.

Smoke Controls consisted of sequentially enrolled participants in the University of Pittsburgh Specialized Centers of Clinically Oriented Research study with ≧30 pack-year smoking histories, but normal spirometry (i.e., forced expiratory volume in 1 second [FEV₁]>80% of normal predicted values and FEV₁/forced vital capacity [FVC]>0.7). These controls also had prospective compilations of smoking histories, diffusing capacity determinations, and high-resolution multi-detector chest CT scans (MDCT) scored using a 0-to-5 point Likert scale by an expert radiologist blinded to subject identities and characteristics (21).

Because <5% of lung transplantation recipients were members of minority racial/ethnic groups, and HLA allele frequencies are often discrepant among races (18), analyses were limited to Caucasians. All subjects gave informed consent under auspices of the University of Pittsburgh Institutional Review Board.

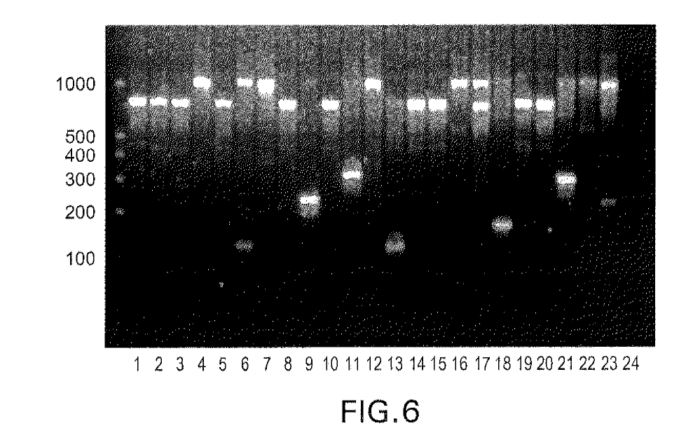

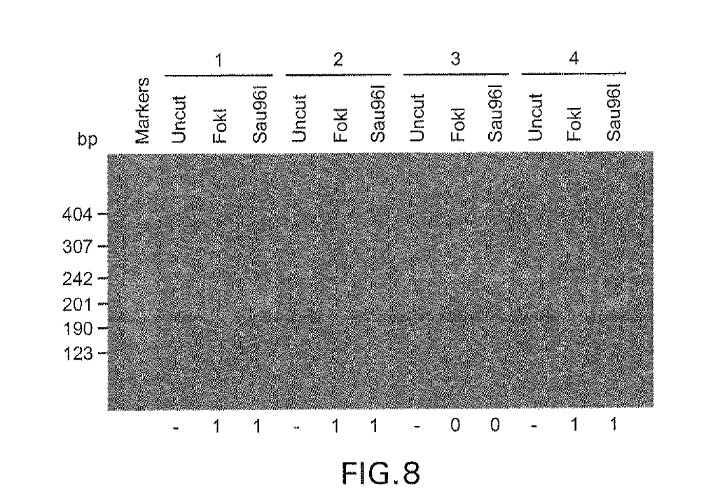

HLA Typing: Comprehensive HLA characterizations of COPD subjects was performed by the Tissue Typing Laboratory at the University of Pittsburgh, using DNA isolated from leukocytes, with sequence specific oligonucleotide probe assays for low/intermediate allele resolutions (Dynal RELI™ SSO, Invitrogen, Carlsbad, Calif.). Polymerase chain reaction (PCR)-sequence specific primer (SSP) amplification was used for high resolution typing and/or resolution of ambiguities (SSP UniTray™, PELFREEZ Clinical Systems, Brown Deer, Wis.). Low/intermediate resolution typing of Smoke Control HLA Class I loci were similarly determined by SSP (Olerup SSP™, Qiagen, Valencia, Calif.) (FIG. 6). High resolution HLA-Cw*07 allele typing of all subjects was performed by SSP (Qiagen) and/or direct genomic sequencing (FIG. 7). DRB 1*15 was delineated among Smoke Controls by PCR-restriction fragment length polymorphism assays (62) (FIG. 8).

Statistical Methods: HLA allele-disease associations were established by chi-square. Comparisons of ordinal data and continuous variables were made by Mann-Whitney. Multivariate analyses of continuous and dichotomous variables were made by logistic regression. Corrections for multiple HLA allele comparisons were made by Bonferroni. Significance was defined as P<0.05.

### Results

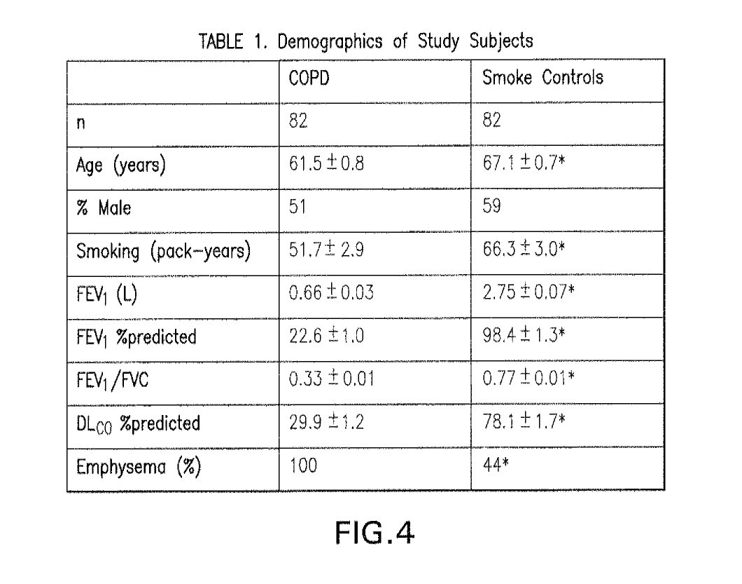

Subjects: Eighty-two (82) end-stage COPD patients had lung transplantations and fulfilled inclusion criteria. Demographic characteristics of these subjects, and the Smoke Controls, are delineated in FIG. 4.

HLA-Cw*07 frequencies: A pilot study in early 2006 indicated that Cw7 was the most frequent HLA Class I allele of COPD patients who had lung transplantations, and was more prevalent in this group than among normal reference populations (18). Since HLA alleles were determined solely by inaccurate serologic methodologies in these subjects, however, this finding was not reported.

Molecular assays have been used for HLA typing of lung transplant candidates with COPD at this institution since May 2006, and these data have been prospectively compiled and used herein. Intergroup comparisons of allele frequencies determined by molecular methodologies confirmed the pilot serologic study, as Cw*07 was over-represented among patients with severe COPD (72%) vs. 39% in Smoke Controls (OR 4.0, CI: 2.1-7.7, P<0.0001).

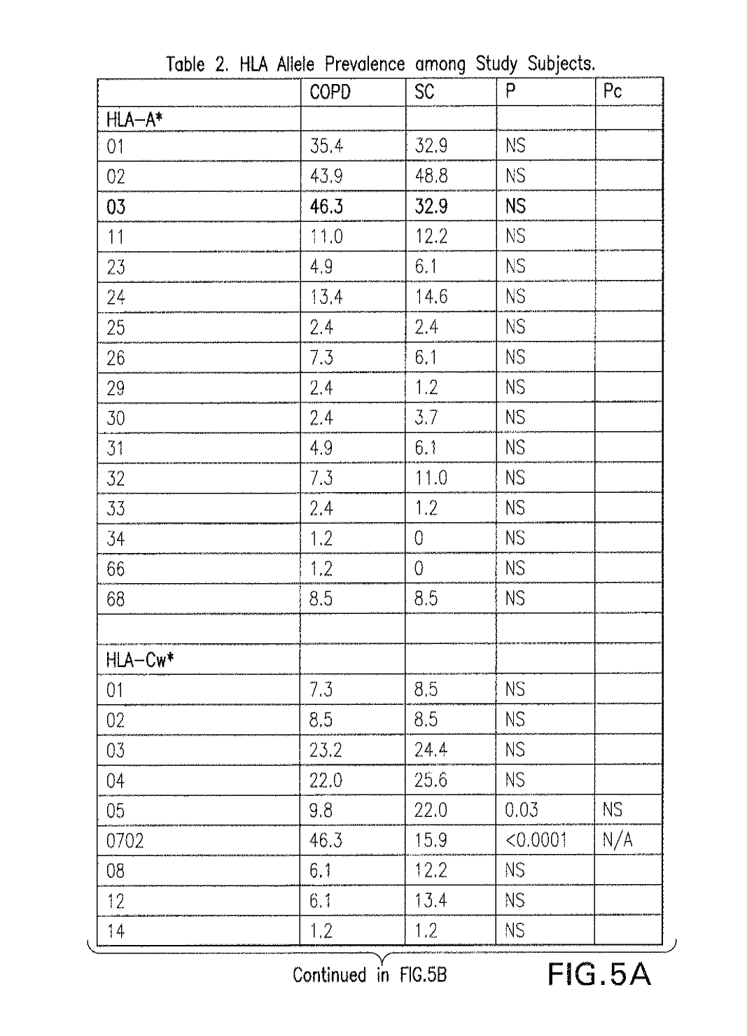

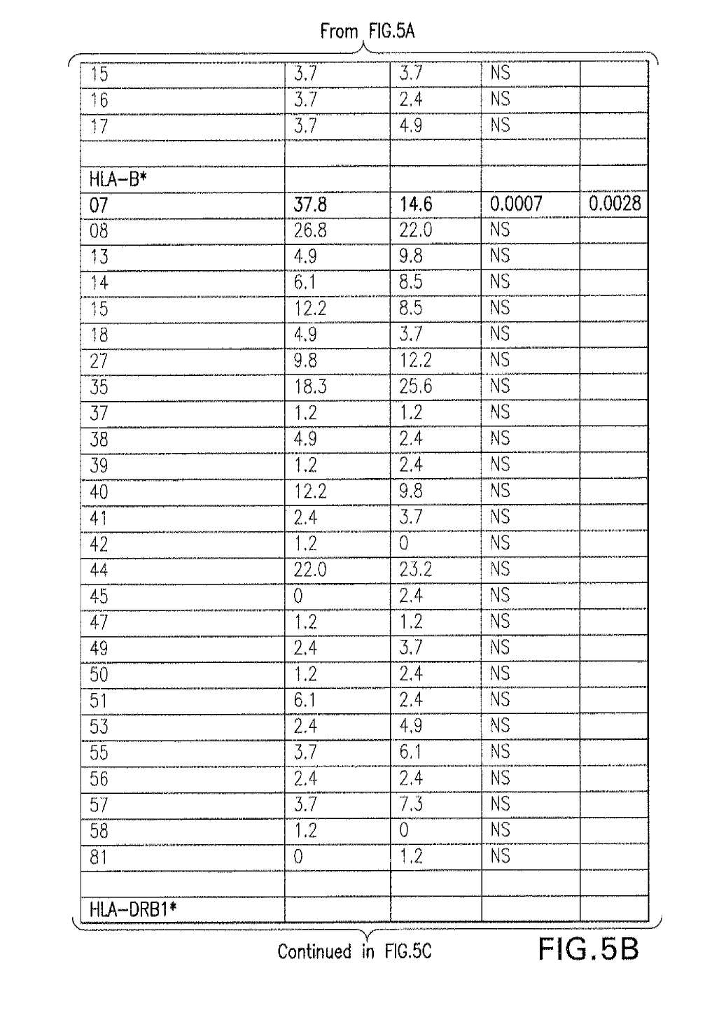

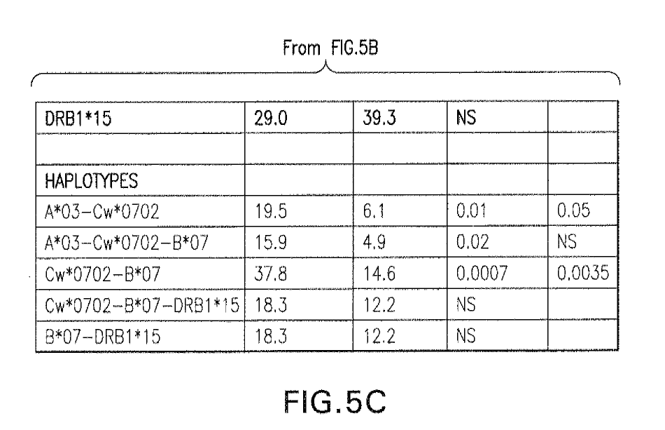

Two distinct Cw*07 alleles (0701 and 0702) are near equally prevalent in Caucasians, however, and these differ in their LD with other HLA alleles (and other proximate genes) (18,15). High-resolution typing showed prevalences of Cw*701 were equivalent among COPD patients and Smoke Controls (30% and 27%, respectively). In contrast, Cw*0702 was significantly over-represented among the COPD subjects compared to Smoke Controls (FIG. 5) (OR 4.6, CI: 2.2-9.5, P<0.0001). This association was not gender-dependent (data not shown).

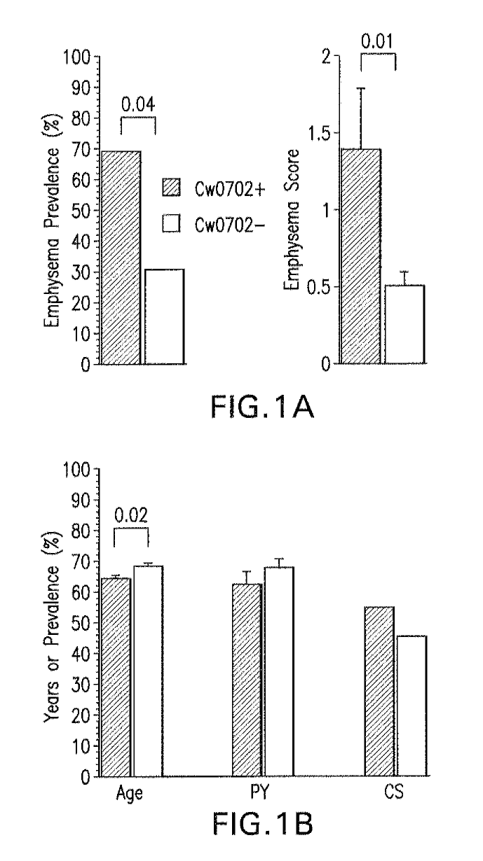

We further assessed the association of Cw*0702 with radiographic emphysema among the Smoke Controls. In contrast, the population with severe COPD typically has far-advanced disease affecting all lung compartments, and thus is less amenable to analyses of distinctive disease subtypes. Despite limited subject numbers, the presence of Cw*0702 was significantly associated with the presence and magnitude of radiographic emphysema (FIG. 1A).

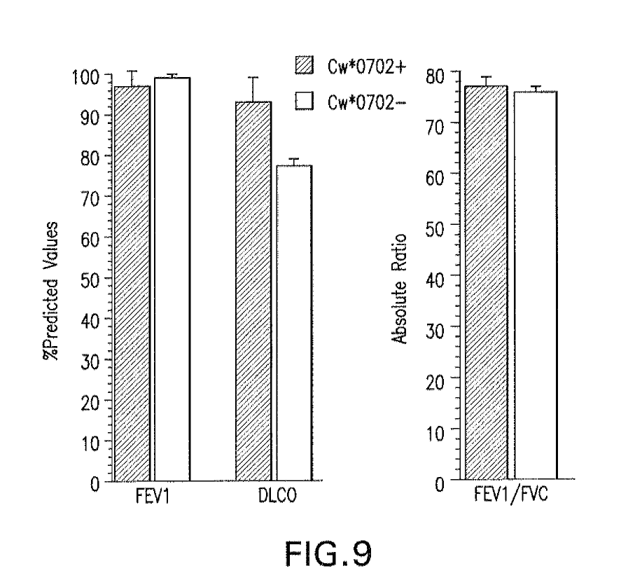

These findings are not attributable to simple demographic differences (FIG. 1B) (see also FIG. 9). Moreover, multivariate logistic regression analyses showed the presence of emphysema was independent of spirometric parameters (e.g., FEV₁% p, P=0.35). Nonetheless, Cw*0702 remained associated with emphysema in these multivariate comparisons (OR: 3.7; CI: 1.0-13.2, P<0.05).

HLA alleles linked with Cw*0702: Cw*0702 is often in LD with other specific, flanking HLA-A (A*02 or A*03) and HLA-B alleles (B*0702), and other proximate non-HLA genes (18,15). These linked variants have been passed through multiple generations as ancestral haplotypes (23). The HLA-A and HLA-B loci of Smoke Controls were typed, and compared to findings in COPD patients, to assess possible haplotype associations with disease.

HLA-B*07 was present in 43 of the 51 subjects with Cw*0702 (84%), was not present in the absence of Cw*0702, and was thus in strong LD with this Cw allele, as reported elsewhere (18, 15). B*07 was also over-represented among COPD patients compared to Smoke Controls (FIG. 5): OR 3.5, CI: 1.7-7.6, P=0.0007, although the magnitude of this disease association was less than that of Cw*0702. HLA-A*03 was also present among 21 (41%) of subjects with Cw*0702, and the haplotype consisting of these two HLA alleles was slightly more prevalent among COPD (FIG. 5). However, this apparent COPD association is almost certainly due to the strong LD with Cw*0702, since the A*03 allele was not independently associated with disease (FIG. 5). None of the other HLA-A or HLA-B alleles were significantly over-represented among COPD (FIG. 5).

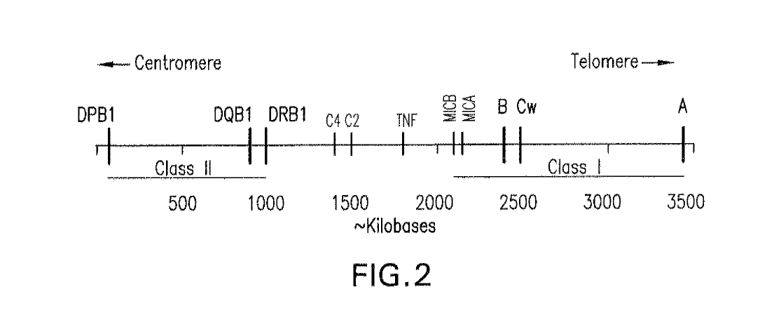

To further define the HLA region most strongly associated with COPD, the possibility was assessed that this disease “hot spot” may extend centromeric (from Cw*0702-B*07) to the HLA Class II-DR locus (FIG. 2). Accordingly, the prevalence of DRB1*15 (DR2) in both groups were compared, given this is the HLA-DR allele most frequently in LD with HLA-Cw*0702-B*07 (18). The strong LD of these alleles was also evident in these study subjects, in that DRB1*15 was also present in 27 (53%) of the Cw*0702 subjects. Nonetheless, there was no significant association of COPD with DRB1*15 (FIG. 5).

HLA-Cw alleles that appear protective for COPD: Frequencies of other Cw alleles were also examined to detect potentially “protective” variants for COPD (FIG. 5). Intergroup analyses were limited to those alleles present in >10% of the Smoke Controls, in order to more productively focus on frequency perturbations with potential biologic/clinical importance.

Several Cw alleles were relatively over-represented among Smoke Controls, including Cw*05, Cw*08 and Cw*12 compared to COPD patients (FIG. 5). However, intergroup differences in frequencies of any individual allele did not reach statistical significance after corrections for multiple comparisons. Nonetheless, the presence of anyone of these three “protective alleles” (Cw*05, Cw*08, or CW*12) was highly associated with a lower probability of disease (OR=0.33; CI: 0.17-0.64, P=0.0012).

Subjects are primarily homozygous for these particular Cw alleles, as only one of the 164 subjects here (a Smoke Control) was heterozygous (Cw*08/Cw*12). Moreover, Cw*05, Cw*08, and Cw*12 appear specifically enriched among the Smoke Controls, rather than a mere mathematical artifact (e.g., due to comparatively less frequent Cw*0702), given that frequencies of the other Cw alleles were similar in both study cohorts (and the normal reference population) (FIG. 5).

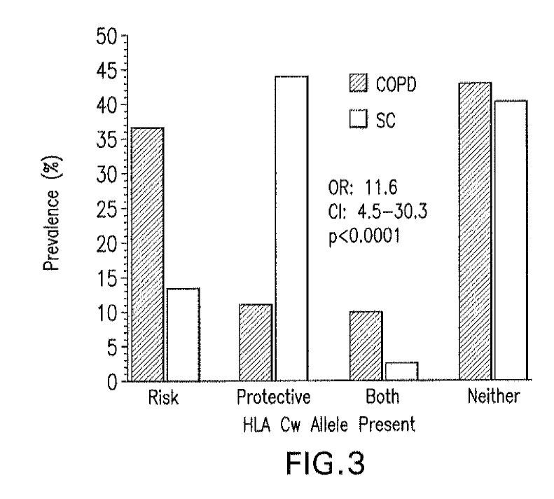

Fifty-eight percent (58%) of subjects here had either the Cw*0702 susceptibility allele or one of the protective alleles (Cw*05, Cw*08, Cw*12) (FIG. 3). Ten subjects (eight COPD, and two Smoke Controls) carried both a risk and a protective Cw allele. The Cw*0702 disease predilection thus appears dominant relative to “protection” of Cw*05, Cw*08, or Cw*12.

Stratifications of subjects according to their Cw alleles, i.e., risk vs. protective vs. neither (and assuming Cw*0702 dominance), reveal very significant associations with COPD. In particular, the presence of a protective Cw allele (and the absence of Cw*0702) yields OR: 0.09, CI: 0.03-0.22 (P<0.0001) for an association with COPD. The converse expression, i.e., any presence of Cw*0702 (with or without a concomitant protective Cw allele) vs. protective or irrelevant Cw alleles is depicted in FIG. 3. There were no meaningful protective associations of any HLA-A or HLA-B allele (FIG. 5).

Cw*0702 Allele Detection By Genomic Sequencing: Specimens positive for Cw*07 (both COPD and Smoke Controls) had additional studies to discriminate between Cw*0701 and Cw*0702 alleles by either/both High Resolution Cw07 SSP (Qiagen), analogous to methodology depicted in FIG. 6, and/or sequencing of the Cw07 genomic segment after amplification by polymerase chain reaction (PCR). Both high resolution SSP and sequence analysis were performed in 28 specimens (equally divided into COPD and Smoke Controls) and results were found to be completely concordant.

In brief, a 207 bp region from exon 2 of the HLA-Cw07 gene was amplified by PCR using forward primer: 5′-TCATCTCAGTGGGCTACGTG-3′ and reverse primer: 5′-CGTCCTCGCTCTGGTTGTA-3′). PCR conditions consisted of 94° C. for 2 min. followed by 35 cycles of 94° C. for 30 sec., 54° C. for 45 sec., and 72° C. for 45 sec., and then a 72° C. incubation for 10 min. PCR products were sequenced using the same primers in an ABI PRISM BigDye Terminator Cycle Sequencing Ready Reaction Kit (Applied Biosystems, Foster City, Calif., USA) in an Applied Biosystems (ABI) 3730×1 DNA Analyzer.

### Discussion

The present findings show the HLA complex is an important genetic element of COPD susceptibility. In particular, HLA-Cw*0702 was highly over-represented among patients with end-stage COPD. The presence of HLA-Cw*0702 among a small proportion of “healthy smokers” was additionally associated with radiographic emphysema in these subjects (FIG. 1A). Furthermore, certain other Cw alleles were enriched among the Smoke Controls, indicative of a genetically-determined “protective” effect. Thus, a susceptibility “hot spot” for COPD appears to be localized within or proximate to the HLA-Cw locus.

A genetic basis for COPD has long been inferred by findings of epidemiologic studies (2,3). Moreover, various other (non-HLA) COPD susceptibility genes have been putatively identified by genome wide association studies (GWAS) (3, 4). However, neither the prevalences of these previously reported disease-candidate polymorphisms, nor the strengths of their associations with COPD, generally approach those seen here with Cw*0702, particularly given the context of this study using a relatively small cohort.

The present data further support concepts that COPD pathogenesis involves interactions between environmental agent(s) (e.g., tobacco smoke) and genetic susceptibility (4), a general disease paradigm also common to many other immunologic disorders, particularly those characterized by autoimmunity (63). Two distinct processes may account for the associations between HLA alleles and immunologic diseases (including COPD).

First, HLA molecules are requisite effectors for presentation of peptide antigens to the T-cells that initiate adaptive immune responses, but each distinct HLA allele has a restricted peptide binding motif (64). Hence, HLA haplotype inheritance pre-determines the finite repertoire of antigens that can evoke T-cell responses in an individual. Although critical for host defense, these adaptive immune responses may be deleterious if, as an example, the antigen is a self-protein (autoantigen), or one that evokes a cross-response to a self-protein (epitope mimicry) (65, 66). In contrast, individuals lacking these specific, “permissive” HLA alleles do not present those particular antigens, and do not initiate the deleterious response(s).

Alternatively, however, overexpression of a specific HLA molecule(s) in a disease cohort may be essentially unrelated to the unique antigen presentations of that HLA per se, but is instead a genetic “marker” denoting the presence of a pathogenic immunomodulatory gene(s) that is in LD with the HLA allele (14, 15).

Pending more complete understanding of COPD pathogenesis, the possibility the Cw*0702 association with COPD is due to a unique antigen presentation by this HLA allele cannot be excluded. However, it seems unlikely the presence of other, specific Cw alleles (i.e., 05, 08, 12) could mitigate pathogenesis solely by presentations of equally unique, “disease-protective” antigens. One theory is that the genetic elements that actually confer susceptibility for or protection from COPD are proximate to and in LD with the respective Cw alleles. Several candidate polymorphic genes are within this region (27), but definitive identification of the responsible variant(s) and mechanism(s) awaits incremental study.

The data presented herein are novel. Only two prior reports describe HLA allele frequencies of COPD patients, and these have been limited to analyses of HLA-A and/or HLA-B loci (28,29). A comparison of HLA-A and -B alleles (by serologic determinations) among small cohorts of never-smokers with airflow obstruction vs. heavy smokers with normal spirometry by Kauffmann, et al. (28), indicated that HLA-B7 may be a susceptibility factor for COPD, independently of smoking. These findings are somewhat concordant with the present data showing associations between HLA-B*07 and COPD due to smoking, although it is likely this relationship is more attributable to the LD of this allele with Cw*0702. Moreover, Kasuga, et al (29), later reported no difference in B*07 frequency (by molecular characterization) between COPD and normal controls. However, those COPD subjects had relatively mild disease and, thus, the findings could be confounded if the COPD risk allele(s) is in lesser frequency among less severely afflicted patients. A COPD susceptibility locus was also localized to chromosome 6 (30), although resolution of specific genes (or regions) was not methodologically feasible. Genomic segments within the HLA complex cannot be readily assessed by GWAS or other global survey assays because of the extreme polymorphism and strong, nonrandom LD within this region (13).

### Example 2

### Alleles Associated with COPD and Alleles Protective Against COPD

HLA allele Cw*07

HLA allele frequencies of patients with severe COPD are abnormal. Given the apparently important role of adaptive immunity (and autoimmunity) in COPD (5,9,12), it was hypothesized that relative over- or under-expressions of particular HLA frequencies may be evident in this disease population, as has been described in many other immunologic disorders (reviewed in 14).

Moreover, because of an appreciation that the magnitude of certain immunologic abnormalities are significantly correlated with COPD severity (9), it was further hypothesized that HLA frequency perturbations may be most prevalent among those patients with the most severe disease. In addition, genetic comparisons of an end-stage COPD cohort to a randomly selected healthy control population might not be as powerful as, instead, using controls with a smoking history, but normal expiratory airflow. This reasoning was based on the assumption that had these Smoke Controls also inherited genes that predisposed to COPD, they would most likely have also developed some airflow obstruction due to their heavy smoking. Thus, the absence of airflow obstruction in these subjects, despite their extensive smoking histories, might reflect they are a population “enriched” for the absence of COPD susceptibility genes, or perhaps instead even be enriched for “disease-protective” genes.

Accordingly, HLA frequencies were compiled (for -A, -Cw, -B, -DR, and -DQ loci) from Caucasian COPD patients who had lung transplantations at the University of Pittsburgh, beginning in May 2006. All of these subjects fulfilled ERS/ATS COPD criteria (19), had severe, end-stage GOLD 4 disease (20) that was attributable solely to smoking (>10 pack years) without any other known causes (e.g., a-1-antitrypsin deficiency, occupational exposures, etc.), and all had severe emphysema on pre-transplant HRCT scans and histology of their explanted lungs.

Controls consist of an equal number of Caucasian subjects sequentially enrolled in the COPD SCCOR study at University of Pittsburgh who have >30 pack-year smoking histories, but normal spirometry (Smoke Controls). Forty-three percent (43%) of these subjects have emphysema on HRCT scans, despite normal spirometry, as ascertained by an expert radiologist who was blinded to subject identities and spirometry results (21). The Smoke Control subjects also had full clinical evaluations and prospective compilations of relevant data given they are subjects in the COPD SCCOR study. the Cw and DR loci of the

Smoke Control subjects have been typed. Demographics of the study populations are delineated in Table 1. Of note, Smoke Controls are older and have smoked more than the COPD subjects.

TABLE 1¹

| | | Age | % | PY | FEV1 | | DLCO |
|---|---|---|---|---|---|---|---|
| | n | (yr) | males | smoke | % p | FEV1/FVC | % p |
| COPD | 82 | 62 + 1* | 47 | 52 + 3* | 25 + 1* | 0.33 + 0.01* | 33 + 1* |
| SC | 82 | 67 + 1 | 53 | 66 + 3 | 98 + 1 | 0.77 + 0.01 | 67 + 1 |

Subject Demographics.
Data here and throughout are expressed as means + SE, and nonparametric tests are used for comparisons.
SC denotes smoke controls;
% p denotes percentages of predicted, normal values.
PY smoke denotes pack-years of smoking.
*p < 0.001 (Mann-Whitney).
¹nAge (yr) % males PY smoke FEV1% p FEV1/FVC DLCO % p
COPD 82 62 + 1* 47 52 + 3* 25 + 1* 0.33 + 0.01* 33 + 1* SC 82 67 + 1 53 66 + 3 98 + 1 0.77 + 0.01 67 + 1*

Genomic DNA was isolated from lung explant tissue or peripheral blood leukocytes that had been stored at −80° C., and was HLA typed using Sequence Specific Primer (SSP) kits (Qiagen, Valencia, Calif.)

It was found that Cw*07 is the single most prevalent HLA allele among the COPD, and is also significantly more frequently expressed in these subjects (72%) than in Smoke Controls (39%) (p<0.0001). Of note, this HLA is present in ˜35-40% of normal Caucasian populations (18).

However, two distinct Cw*07 alleles (0701 and 0702) are prevalent in Caucasians, and these two differ in their LDs with other HLA alleles (and other proximate genes), and their respective antigen epitope presentation motifs. Accordingly, high resolution SSP typing was performed to distinguish these alleles. These findings show that the intergroup difference of Cw*07 is actually due to an increased prevalence of Cw*0702 among the COPD (36%) vs. 14.6% in the Smoke Controls 0.0003). The Odds Ratio (OR) and 95% Confidence Intervals (CI) for the association between Cw*0702 and COPD is 3.3, CI: 1.5-7.5. In contrast, the frequency of Cw*0701 is near equal in both populations (27% and 28%). The association between Cw*0702 and COPD is ˜equivalent, and significant, among both male and female subjects (data not shown). The prevalence of Cw*0702 among the COPD here is also much greater than in random, healthy normals (18).

Cw*0702 may also be associated with clinical manifestations in “healthy smokers”. Although the highly significant differences here are very unlikely attributable to chance, additional evidence was sought that Cw*0702 is associated with pathologic processes. Of particular interest was to determine if the presence of Cw*0702 among Smoke Controls correlated with other clinical features. It was anticipated that these subjects would be the least “noisy” cohort for this initial correlation, since the end-stage COPD patients invariably had extremely severe clinical manifestations. Even though the number of Smoke Controls is small

(particularly those that express Cw*0702), several HLA associations have been found: Smoke Controls who are positive for Cw*0702 have a greater prevalence than the cohort that did not inherit this allele in terms of frequent coughing (42% vs. 19%, p=0.07), wheezing with viral upper respiratory tract infections (67% vs. 36%, p=0.04) and have worse symptom scores (a domain of the St. George Respiratory Questionnaire (22)).

In addition, those Smoke Controls expressing Cw*0702 tend to have slightly lower FEV₁% p than the controls who did not inherit this gene (88% vs. 93%, respectively) and have greater increases of FEV₁ with bronchodilators (11.3+4.5% vs. 6.9+1.4%, respectively), implying an association between Cw*0702 and a predilection for airway reactivity among “healthy” subjects with smoking histories.

Other Genes are Linked with the Cw*0702 Risk Allele

What other genes are linked with the Cw*0702 risk allele? Cw*0702 is typically in LD with other specific HLA alleles (and flanking, non-HLA genes). These linked genes have been passed through multiple generations from long distant progenitors, and are referred to as ancestral haplotypes (23). Two Cw*0702 ancestral haplotypes are most frequently (and nearly equally) expressed among Caucasians (18) and both of these have been associated with immunologic diseases (15,24,25), i.e., A*02, Cw*0702, B*0702, DRB1*1501, DQB1*0602 or

HLA A*03, Cw*0702, B*0702, DRB1*1501, DQB1*0602. The latter haplotype (A*03 . . . DQB1*0602) is especially associated with autoimmunity, and has recently been completely sequenced (15).

HLA-B*07 is present in 90% of the COPD here who are positive for Cw*0702, and is not expressed independently of Cw*0702 in this cohort. This frequency is >2-fold the B*07 prevalence in normal Caucasian populations (18). The B*07 allele that is most frequently in LD with Cw*0702 is B*0702 (18). Therefore, the B*07 allele that seems overexpressed among the COPD here is almost certainly B*0702 (confirmation by high resolution typing is pending). Half of the COPD subjects here who express both Cw*0702 and B*07 also express A*02, and the other half express A*03. Until we finish typing, we cannot definitively ascertain the telomeric “end” of the ancestral haplotype(s) that seems most overexpressed among the

COPD. At least for now, however, these data indicate the Cw*0702-B*0702 region is likely the predominant HLA class I “hot spot”.

Loci more centromeric to the Cw*0702 (and likely B*0702) region study of HLA have also been studied, namely the HLA Class II segment. DRB1*15 is the most frequently expressed DR allele among the COPD subjects here (29%). DRB1*15 is often in LD with Cw*0702-B*0702 (18). Assays for the presence of DRB1*15 in Smoke Controls were performed, and found the allele prevalence in this cohort (39%) is comparable to the COPD. Thus, the COPD “hot spot” may not extend very centromeric to the Cw*0702-B*0702 region.

However, while DRB1*15 does not appear to be a risk factor for COPD per se, this allele is nonetheless associated with the presence of emphysema (without airflow obstruction) among the Smoke Controls: Emphysema is present in 60% of DRB1*15 positive Smoke Controls, compared to 32% prevalence among the cohort who did not inherit this allele (p=0.017). The Odds Ratio (OR) and 95% Confidence Intervals (CI) for the association between DRB1*15 and emphysema is 3.2, CI: 1.2-8.3. Among the group of Smoke Control subjects who have emphysema (n=33), those with DRB1*15 have worse emphysema scores than the subjects who did not inherit this allele (1.7+/−0.2 vs. 1.3+/−0.2, p=0.05). These data suggest that airflow obstruction and emphysema could, at least to some degree, have partially independent underlying genetic determinants. This putative hypothesis may also be consistent with suggestions elsewhere that airflow obstruction per se and emphysema may, at least to some degree, be distinct disease phenotypes (26,27).

### Protective HLA Alleles

Do other HLA alleles protect smokers from severe COPD? Complete HLA-Cw allele typing of our study cohort also revealed that Cw*05 is much less prevalent among COPD (10%) than in Smoke Controls (22%), resulting in OR 0.38, 95% CI: 0.16-0.94 (p=0.03) for concordances of this allele with severe COPD. The prevalence of Cw*05 in the Smoke Controls is also greater than frequencies of this allele among normal populations (18), perhaps suggesting an “enrichment” among the former. Altogether, these data imply that Cw*05, or a gene(s) in proximity (and LD) to it, may confer protection from COPD. In addition, Cw*08 and Cw*12 also seem overexpressed among the Smoke Controls (12% and 13%, respectively), relative to both normal populations (18), and the COPD (6% frequencies for each allele). These three Cw alleles also segregate independently, in that only one of the 164 subjects here (a Smoke Control) is heterozygous for two of these alleles (Cw*08 plus Cw*12). The presence of any one of the three “protective alleles” (Cw*05, Cw*08, or Cw*12) among the aggregate study subjects here is highly associated with freedom from disease, with OR 0.32; 95% CI: 0.17-0.64, p=0.0012.

Approximately half (46%) of our total study population express either the susceptibility allele (Cw*0702) or one of the three protective Cw alleles (05, 08, 12). Six subjects (4 COPD, and 2 Smoke Controls) co-expressed both the risk and one of the protective alleles. Thus, and although obviously based on very small numbers, the predilection for disease associated with Cw*0702 here appears to possibly be dominant relative to protective effects of Cw*05, Cw*08, or Cw*12.

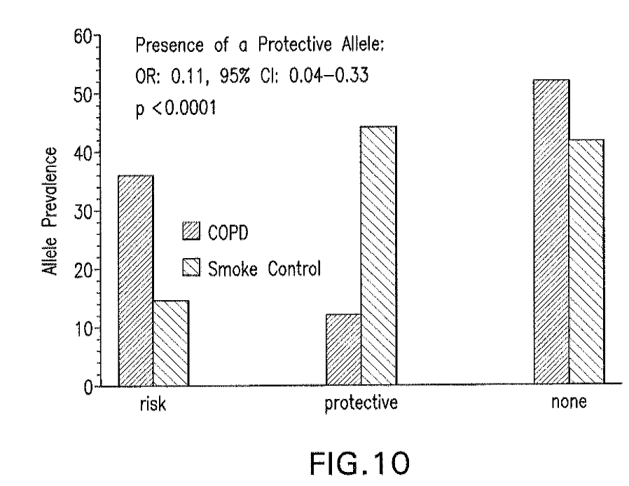

In the aggregate, these findings show that even with our preliminary HLA allele comparisons so far, limited primarily to the Cw locus, our early data suggest we can stratify “risks for COPD” in half the subject population. Moreover, attributions of disease-specific risks in our subjects, now segregated solely on the basis of their Cw alleles, i.e., risk vs. protective vs. neither (and assuming Cw*0702 dominance), reveal very significant associations with COPD (FIG. 10): the OR for the presence of a COPD protective allele (vs. Cw*0702) is 0.11, CI 0.04-0.33. Conversely expressed, the presence of Cw*0702 (in the context of this Cw stratification paradigm) yields OR 9.0; 95% CI: 3.0-25.0 (p<0.0001) for the association with severe, end-stage COPD.

There have been two prior reports specifically examining HLA frequencies among COPD patients, and these have been limited to analyses of HLA-A and/or HLA-B loci. A comparison of HLA-A and -B allele frequencies (by comparatively inaccurate serologic determinations) in a small cohort by Kauffmann et al. (28) indicated that HLA-B7 may be a susceptibility factor for COPD (p=0.05). These findings were later disputed by Kasuga, et al (29), who reported no difference in B07 frequency between COPD and normal controls. However, their COPD subjects had relatively mild disease (GOLD 2) and, thus, these conclusions could be falsely negative if COPD “risk alleles” are actually in greatest frequency among the most severely afflicted. Moreover, they did not type any HLA alleles other than B07. Another recent report localized a COPD susceptibility locus to chromosome 6 (30), although resolution of specific genes (or a finite genomic “hot spot”) was not methodologically feasible.

### Example 3

### Determination of Haplotypes Associated with IPF

HLA frequencies in other chronic lung diseases including idiopathic pulmonary fibrosis (IPF) have been examined. In IPF, the strongest association was found with DRB1*1501, although associations between IPF and Cw*07 and B*07 were also found which could be explained by strong linkage disequilibrium. In this case, the DR allele was defined by high resolution methodology, which found that this allele was DRB1*1501 and in linkage disequilibrium with DQB1*0602.

### BIBLIOGRAPHY AND LITERATURE CITED

1. Burrows B, Niden A H, Barclay W R, Kasik J E. Chronic obstructive pulmonary disease II. Relationships of clinical and physiological findings to the severity of airways obstruction. Am Rev Respir Dis, 1965; 91:665-678
2. Kurzius-Spencer M, Sherrill D L, Holberg C J, Martinez F D, Lebowitz M D. Familial correlation in the decline of forced expiratory volume in one second. Am J Respir Crit Care Med, 2001; 164:1261-1265
3. Molfino N A. Genetics of COPD. Chest, 2004; 125:1929-1940
4. Sandiford A J, Silverman E K. Chronic obstructive pulmonary disease. 1: Susceptibility factors for COPD the genotype-environment interaction. Thorax, 2002; 57:736-741
5. Feghali-Bostwick C A, Gadgil A S, Otterbein L E, Pilewski J M, Stoner M W, Csizmadia E, Zhang Y, Sciurba F C, Duncan S R. Autoantibodies in patients with chronic obstructive pulmonary disease. Am J Resp Critical Care Med 2008; 177:156-163
6. Nagata Y, Hanagiri T, Takenoyama M, Fukuyama T, Mizukami M. Identification of the HLA-Cw*0702-restricted tumor-associated antigen recognized by a CTL clone from a lung cancer patient. Clin Cancer Res, 2005; 11:5265-5272
7. Hong J J, Reynolds M R, Mattila T L, Hage A, Watkins D I, Miller C J, Skinner P J. Localized populations of CD8 MHC I tetramer SIV-specific T cells in lymphoid follicles and genital epithelium. Plos One 2009; 4:e4131
8. Hahn M, Nicholson M J, Pyrdol J, Wucherpfennig K W. Unconventional topology of self peptide-major histocompatibility complex binding by a human autoimmune T cell receptor. Nat Immunol, 2005; 6:434-435
9. Zhu X, Gadgil A S, Givelber R, George M P, Stoner M W, Sciurba F C, Duncan S R. Peripheral T-cell functions correlate with the severity of chronic obstructive pulmonary disease. J. Immunol 2009; 182:3270-3277
10. Finkelstein R, Fraser R S, Ghezzo H, Cosio M G. Alveolar inflammation and its relation to emphysema in smokers. Am J Respir Crit Care Med 1995; 152:1666-72
11. Di Stefano A, Capelli A, Lusuardi M, Balbo P, Vecchio C, Maestrelli P, Mapp C E, Fabbri L M, Donner C F, Saetta M. Severity of airflow limitation is associated with severity of airway inflammation in smokers. Am J Respir Crit Care Med 1998; 158:1277-1285
12. Hogg J C, Chu F, Utokaparch S, Woods R, Elliot W M, Buzatu L, Cherniack R M, Rogers R M, Sciurba F C, Coxson H O, Pare P D. The nature of small-airway obstruction in chronic obstructive pulmonary disease. N Engl J Med 2004; 350:2645-53
13. Traheme J A, Horton R, Roberts A N, Miretti M M, Huries M E, Stewart C A, Ashurst J L, Atrazhev A M, Goggill P, Palmer S, Almeida J, Sims S, Wilming L G, Rogers J, de Jong P J, Carrington M, Elliot J F, Sawcer S, Todd J A, Trowsdale J, Beck S. Genetic analysis of completely sequenced disease-associated MHC haplotypes identifies shuffling of segments in recent human history. PLoS Genetics, 2006; 2:0081
14. de Bakker P I, McVean G, Sabeti P C, Miretti M M, Green T, Marchini J, Ke Y, Monsuur A J, Whittaker P, Delgad M, Morrison J, Richardson A, Walsh E C, Gao X, Galver L, Hart J, Hafler D A, Bericak-Vance M, Todd J A, Daly M J, Trowsdale J, Wijmenga C, Vyse T J, Beck S, Murray S S, Carrington M, Gregory S, Deloukas P, Rioux J D. A high-resolution HLA and SNP haplotype may for disease association studies in the extended human MHC. Nature Genetics, 2006; 38:1166-1172
15. Stewart C W, Horton R, Allcock R J N, Ashurst J L, Atrazhev A M, Coggill P, Dunham I, Forbes S, Halls K, Howson J M M, Humphray S J, Hunt S, Mungall A J, Osoegawa K, Palmer S, Roberts A N, Rogers J, Sims S, Wang Y, Wilming L G, Elliot J F, de Jong P J, Sawcer S, Todd J A, Trowsdal J, Beck S. Complete MHC haplotype sequencing for common disease gene mapping. Genome Res, 2004; 14:1176-1187
16. Huang S L, Su C H, Chang S C. Tumor necrosis factor-&#61537; gene polymorphism in chronic bronchitis. Am J Respir Crit Care Med 1997; 156:1436
17. Higham M A, Pride N B, Alikhan A, Morrell N W. Tumour necrosis factor-alpha gene promoter polymorphism in chronic obstructive pulmonary disease. Eur Respir J, 2000; 15:281-284
18. National Bone Marrow Donor Program. Haplotype Frequencies. bioinformatics.nmdp.org. Last accessed Apr. 4, 2009
19. Global Initiative for Chronic Obstructive Lung Disease. Global strategy for the diagnosis, management, and prevention of chronic obstructive pulmonary disease. www.goldcopd.com. Date last assessed Mar. 10, 2009
20. Celli B R, MacNee W; ATS/ERS. Standards for the diagnosis and treatment of patients with COPD: a summary of the ATS/ERS position paper. Eur Respir J, 2004; 23:932-946
21. Wilson D O, Weissfeld J L, Balkan A, Schragin J G, Fuhrman C R, Fisher S N, Wilson J, Leader J K, Siegfried J, Shapiro S D, Sciurba F C. Association of radiographic emphysema and airflow obstruction with lung cancer. Am J Respir Crit Care Med 2008; 178(7):738-44.
22. Jones, P. W., F. H. Quirk, C. M. Baveystock, P. Littlejohns P. A self-complete measure of health status for chronic airflow limitation: the St. George's Respiratory Questionnaire. Am. Rev. Respir. Dis. 1992; 145:1321-1327
23. Dawkins R L, Christiansen F T, Kay P H, Garlepp M, McCluskey J, Hollingsworth P N, Zilko P J. Disease associations with complotypes, supratypes and haplotypes. Immunol Rev 1983; 70:1-8
24. Hall F C, Bowness P. HLA and disease: From molecular function to disease association? In HLA and MHC: Genes, molecules, and function (eds. Browning M J, McMichael A J), 1996, pp 353-381. BIOS Scientific Publishers Ltd, Oxford, UK.
25. Warrens A, Lechler R. HLA in health and disease. 1999, Academic Press, San Diego, Calif.
26. Sciurba, F C. Physiologic similarities and differences between chronic obstructive pulmonary disease (COPD) and asthma. Chest 2004; 126(2 suppl):117S-124S; discussion 159S-161S.
27. Sciurba F C. COPD Phenotypes: The answers are in the noise. J of COPD, 2007; 4(4):301-302.
28. Kauffmann F, Kleisbauer Cambon-De-Mouzon A, Mercier P, Constans J, Blanc M, Rouch Y, Feingold N. Genetic markers in chronic air-flow limitation. Am Rev Resp Dis, 1983; 127:283-289
29. Kasuga I, Ruan J, Connett J E, Anthonisen N R, Sandford A. J. Lack of association of human leukocyte antigen-B7 with COPD and rate of decline in lung function. Resp Med, 2005; 99:1528-1533
30. Joost O, Wilk J B, Cupples L A, Harmon M, Shearman A M, Baldwin C T, O'Connor G T, Myers R H, Gottlieb D J. Genetic loci influencing lung function: a genome-wide scan in the Framingham study. Am J Resp Crit Care Med 2002; 165:795-799.
31. American Thoracic Society. 2000. Idiopathic pulmonary fibrosis: diagnosis and treatment. International consensus statement. American Thoracic Society (ATS), and the European Respiratory Society (ERS). Am. J. Respir. Grit. Gare Med. 161:646-664.
32. Selman, M., V. J. Thannickal, A. Pardo, D. A. Zisman, F. J. Martinez, and L. P. Lynch, III. 2004. Idiopathic pulmonary fibrosis: pathogenesis and therapeutic approaches. Drugs 64:406-430.
33. Collard H. R., T. E. King, B. B. Bartelson, J. S. Vourlekis, M. I. Schwartz and K. K. Brown. Changes in clinical and physiologic variable predict survival in idiopathic pulmonary fibrosis. Am J Respir Grit Gare Med 2004; 169:538-542
34. Kurosu K., Y. Takiguchi, O. Okada, N. Yumoto, S. Seiichiro, Y. Tada, Y. Kasahara, N. Tanabe, K. Tatsumi, M. Weiden, W. N. Rom, and T. Kuriyama. Identification of annexin 1 as a novel autoantigen in acute exacerbation of idiopathic pulmonary fibrosis. J Immunol, 2008; 181: 756-767.
35. Takahashi T., I. Wada, Y. Ohtsuka, M. Munakata, Y. Homm, and Y. Kuroki. Autoantibody to alanyitRNA synthetase in patients with idiopathic pulmonary fibrosis. Respirology 2007; 12:642-653.
36. Magro, C. M., W. J. Waldman, D. A. Knight, J. N. Allen, T. Nadasdy, G. E. Frambach, P. Ross, and C. B. Marsh. 2006. Idiopathic pulmonary fibrosis related to endothelial injury and antiendothelial cell antibodies. Hum. Immunol. 67:284-297.
37. Dobashi, N., J. FUjita, M. Murota, Y. Ohtsuki, I. Yamadori, T. Yoshinouchi, R. Ueda, S. Bandoh, T. Kamei, M. Nishioka, T. Ishida, and J. Takahara. 2000. Elevation of anti-cytokeratin 18 antibody and circulating cytokeratin 18: anti-cytokeratin 18 antibody immune complexes in sera of patients with idiopathic pulmonary fibrosis. Lung 178:171-179.
38. Wallace W. A., J. A. Schofield, D. Lamb, S. E. Howie. Localization of a pulmonary autoantigen in cryptogenic fibrosing alveolitis. Thorax 1994; 49: 1139-1145.
39. Pignatti P., G. Brunetti, D. Moretto, M. R. Yacoub, M. Fiori, B. Balbi, A Balestrino, G. Cervio, S. Nava, and G. Moscato. Role of the chemokine receptors CXCR3 and CCR4 in human pulmonary fibrosis. Am J Respir Crit Care Med 2006; 173:310-317.
40. Papiris S. A, A Kollintza, M. Karatza, E. D. Manali, C. Sotiropoulou, J. Milic-Emili, C. Roussos, Z. and Daniil. CD8+ T lymphocytes in broncholaveolar lavage in idiopathic pulmonary fibrosis. J Inflamm (Lond) 2007; 4:14-18
41. Marchal-Somme J., Y. Uzunhan, S. Marchand-Adam, D. Valeyre, V. Soumelis, B. Crestani, and P. Soler. Cutting edge: non-proliferating mature immune cells form a novel type of organizing lymphoid structure in idiopathic pulmonary fibrosis. J Immunol 2006; 176:5735-5739
42. Homolka, J., M. W. Ziegenhagen, K. I. Gaede, P. Entzian, G. Zissel, and J. Muller-Quernheim. 2003. Systemic immune cell activation in a subgroup of patients with idiopathic pulmonary fibrosis. Respiration. 70:262-269
43. Shimizudani, A, H. Murata, H. Keino, S. Kojo, H. Nakamura, Y. Morishima, I. Sakamoto, M. Ohtsuka, K. Sekisawa, M. Sumida, I. Sumida, and I. Matsuoka. Conserved CDR 3 region of T cell receptor BV gene in lymphocytes from bronchoalveolar lavage fluid of patients with idiopathic pulmonary fibrosis. Clin. Exp. Immuno/2002; 129:140-149.
44. Feghali-Bostwick C. A, C. G. Tsai, V. G. Valentine, S. Kantrow, M. W. Stoner, J. M. Pilewski, A Gadgil, M. P. George, K. F. Gibson, A M. K. Choi, N. Kaminski, Y. Zhang, and S. R. Duncan. Cellular and humoral autoreactivity in idiopathic pulmonary fibrosis. J Immuno/2007; 179: 2592-9
45. Rosas I. O., P. Ren, N. A Avila, C. K. Chow, I. J. Franks, W. D. Travis, J. P. McCoy, R. M. May, H. P. Wu, D. M. Nguyen, M. Arcos-Burgos, S. D. MacDonald, and B. R. Gochuico. Early interstitial lung disease in familial pulmonary fibrosis. Am J Resp Crit Care Med 2007; 176:698-705.
46. Marchal-Somme J., Y. Uzunhan, S. Marchand-Adam, M. Kambouchner, D. Valeyre, B. Crestani, and P. Soler. Dendritic cells accumulate in human fibrotic interstitial lung disease. Am J Respir Crit Care Med 2007; 176:1007-1014.
47. Bitterman P B, Rennard S I, Keogh B A, Wewers M D, Adelberg S, Crystal R G. Familial idiopathic pulmonary fibrosis. Evidence of lung inflammation in unaffected family members. N Eng J Med 1986; 314:1343-7
48. Vallejo A N., C. M. Weyand, and J. J. Goronzy. T-cell senescence: a culprit of immune abnormalities in chronic inflammation and persistent infection. Trends Mol Med 2004; 10: 119-124.
49. Studer S. M., M. P. George, X. Zhu, Y. Song, V. G. Valentine, M. W. Stoner, J. Sethi, C. Steele, and S. R. Duncan. CD28 Down-regulation on CD4 T-cells is a marker for graft dysfunction in lung transplant recipients. Am J Resp Crit Care Med 2008; 178:765-773.
50. Morell, P. Personal Communication
51. Varpela E., Tiilkainen A, Varpela M, Tukiainen P. High prevalences of HLA-B15 and HLA-Dw6 in patients with cryptogenic fibrosing alveolitis. Tissue Antigens 1979; 14:68-71
52. Turton C W G, Morris L M, Lawler S O, Turner-Warwick M. HLA in cryptogenic fibrosing alveolitis. Lancet 1978; 1(8064507-8
53. Evans C. HLA antigens in diffuse fibrosing alveolitis. Thorax 1976; 31:483-5
54. Strimlan C V, Taswell H F, DeRemee R A, Kueppers F. HL-A antigens and fibrosing alveolitis. Am Rev Resp Dis 1977; 1120-1
55, Fulmer J D, Sposovska M S, von Gal E R, Crystal R G, Mittal K K. Distribution of HLA antigens in idiopathic pulmonary fibrosis. Am Rev Resp Dis 1978; 118:141-47
56. Libby O M, Gibofsky A, Fotino M, Waters S J, Smith J P. Immunogenetic and clinical findings in idiopathic pulmonary fibrosis. Am Rev Resp Dis 1983; 127:618-22
57. Falfa-Valencia R, Camarena A, Juarez A, Becerril C, Montano M, Cisneros J, Mendoza F, Granados J, Pardo A, Selman M. Major histocompatibility complex and alveolar epithelial apoptosis in idiopathic pulmonary fibrosis. Hum Genetics, 2005; 118:235-244
58 Mannino O M COPD: epidemiology, prevalence, morbidity and mortality, and disease heterogeneity. Chest, 2002, 121:121 S-6S
59. O'Shaughnessy T C, Ansari T W, Barnes N C, Jefferey P K. Inflammation in bronchial biopsies of SUbjects with chronic bronchitis: Inverse relationship of CD8+ T-lymphocytes with FEV1. Am. J. Respir Crit. Care Med, 1997, 155:852-7
60. Cosio M G, Saetta M, Agusti A. Immunologic aspects of chronic obstructive pulmonary disease. N Engl J Med, 2009, 360:2445-2454
61. Grumelli S., Cony D B, Song L Z, et al. An immune basis for lung parenchymal destruction in chronic obstructive pulmonary disease and emphysema. PLoS Med, 2004, 1:75-83
62. Feghali-Bostwick C, Medsger T A, Wright T M. Analysis of systemic sclerosis in twins reveals low concordances for disease and high concordance for the presence of antinuclear antibodies. Arthritis Rheum, 2003, 48:1956-6
63. Marrack P, Kappler J, Kotzin B L. Autoimmune disease: why and where it occurs. Nat Med 2001, 8:899-905
64. Rudolph M G, Stanfield R I, Wilson I A. How TCRs bind MHCs, peptides, and coreceptors. Annu Rev Immunol, 2006, 24:419-466
65. Oldstone M B. Molecular mimicry, microbial infection and autoimmune disease: evolution of the concept. Curr Top Microbiollmmunol. 2005, 296:1-17
66. Vanderlugt C I, Miller S D. Epitope spreading in immune mediated diseases: implications for immunotherapy. Nat Rev Immunol, 2002, 2:85-94
67. Horton R, Wilming I, Rand V, et al. Gene map of the extended human MHC. Nat Rev Genet, 2004, 12:889-899
68. Feghali-Bostwick C A, Gadgil A S, Otterbein I E, et al. Autoantibodies in patients with chronic obstructive pulmonary disease. Am J Resp Critical Care Med, 2008, 177:156-163
69. Lee, S-H., S. Goswami, A. Grudo, et al. Antielastin autoimmunity in tobacco smoking-induced emphysema. Nat Med, 2007, 13:567-9
70. Hankinson, Odencrantz J R, Fedan K B. Spirometric reference values from a sample of the general U.S. population. Am. J. Respir. Crit. Care Med, 1999, 159: 179-87
71. Miller A, Thornton J C, Warshaw R, Anderson H, Teirstein A S, Selikoff I J. Single breath diffusing capacity in a representative sample of the population of Michigan, a large industrial state. Predicted values, lower limits of normal, and frequencies of abnormality by smoking history. Am Rev Respir Dis, 1983, 127:270-277.
72. Robinson J, Waller M J, Parham P, de Groot N, Bontrop R, Kennedy L J, Stoehr P, Marsh S G E. IMGT/HLA and IMGT/MHC: sequence databases for the study of the major histocompatibility complex. Nucleic Acids Research (2003), 31:311-314.
73. Robinson J, Waller M J, Parham P, Bodmer J G, Marsh S G E. IMGT/HLA—a sequence database for the human major histocompatibility complex. Nucleic Acids Research (2001), 29:210-213.
74. Robinson J, Malik A, Parham P, Bodmer J G, Marsh S G E. IMGT/HLA—a sequence database for the human major histocompatibility complex. Tissue Antigens (2000), 55:280-287.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Various references are cited herein, the contents of which are hereby incorporated by reference in their entireties.

What is claimed is:

1. A method for diagnosing chronic obstructive pulmonary disease in a human subject comprising determining whether the subject carries an HLA allele selected from the group consisting of Cw*07, Cw*0702, HLA-B*07, HLA-B*0702, HLA-A*03 and DRB1*1501, wherein detecting the at least one HLA allele indicates that the subject has an increased risk of developing chronic obstructive pulmonary disease compared to a subject that does not have the HLA allele.

2. The method of claim 1, wherein the at least one HLA allele is Cw*0702.

3. The method of claim 1, wherein the HLA allele is detected in a sample selected from the group consisting of leukocytes and lung tissue biopsy.

4. The method of claim 1, wherein the detecting is selected from the group consisting of nucleic acid detection and immunodetection.

5. The method of claim 4, wherein the detecting is nucleic acid detection and the nucleic acid is genomic DNA or RNA.

6. The method of claim 4, wherein the detecting is nucleic acid detection and the nucleic acid detection is an assay selected from the group consisting of polymerase chain reaction (PCR), quantitative PCR, nucleic acid sequenceing, and nucleic acid microarray analysis.

7. A method for diagnosing idiopathic pulmonary fibrosis in a human subject comprising determining whether the subject carries an HLA allele selected from the group consisting of Cw*07, Cw*0702, HLA-B*07, HLA-B*0702, HLA-A*03, DRB1*1501 and DQB1*0602, wherein detecting the at least one HLA allele indicates that the subject has an increased risk of developing idiopathic pulmonary fibrosis compared to a human or mammal that does not have the HLA allele.

8. The method of claim 7, wherein the at least one HLA allele is DRB1*1501.

9. The method of claim 7, wherein the HLA allele is detected in a sample selected from the group consisting of leukocytes and lung tissue biopsy.

10. The method of claim 7, wherein the detecting is selected from the group consisting of nucleic acid detection and immunodetection.

11. The method of claim 10, wherein the detecting is nucleic acid detection and the nucleic acid is genomic DNA or RNA.

12. The method of claim 10, wherein the detecting is nucleic acid detection and the nucleic acid detection is an assay selected from the group consisting of polymerase chain reaction (PCR), quantitative PCR, nucleic acid sequenceing, and nucleic acid microarray analysis.

13. A method of diagnosing COPD or IPF in an individual, the method comprising:
(a) amplifying at least one HLA allele selected from the group consisting of Cw*07, Cw*0702, HLA-B*07, HLA-B*0702, HLA-A*03, DRB1*1501 and DQB1*0602, or fragments thereof, in a sample from the individual;
(b) detecting the amplified nucleic acid portions; and
(c) diagnosing the individual as having an increased risk of developing COPD or IPF.

14. The method of claim 13, further comprising, where the individual is diagnosed as having an increased risk of developing COPD or IPF, administering inhaled corticosteroids, macrolides, or statins to the individual.

15. The method of claim 13, further comprising, where the individual is diagnosed as having an increased risk of developing COPD or IPF, administering a pulmonary function test for monitoring progression of COPD or IPF.

16. The method of claim 13, further comprising, where the individual is diagnosed as having an increased risk of developing COPD or IPF, initiating smoking cessation efforts.

17. A method of evaluating whether an individual is at risk of developing COPD, comprising:
(a) amplifying at least one HLA allele selected from the group consisting of Cw*05, Cw*08 and Cw*12, or fragments thereof, in a sample from the individual;
(b) detecting the amplified nucleic acid portions; and
(c) identifying the individual as having a reduced risk of developing COPD.

18. A kit for detecting an HLA allele, wherein the kit comprises a plurality of oligonucleotide primers, each of which is capable of specifically hybridizing to a desired HLA allele region.

19. A kit according to claim 18, wherein each of the plurality of oligonucleotide primers is immobilized on a solid surface or support.

20. A kit according to claim 18, further comprising a polymerase and nucleotide bases.

21. A kit according to claim 20, wherein the nucleotide bases are detectably labeled.
